(19) United States
(12) Patent Application Publication
(10) Pub. No.: US 2011/0293664 A1
(43) Pub. Date: Dec. 1, 2011

(54) CHLAMYDIA ANTIGENS AND USES THEREOF

(75) Inventors: Kenya Cohane, Worcester, MA (US); Michele Picard, Dover, MA (US); Todd Gierahn, Brookline, MA (US); Jessica Flechtner, Maynard, MA (US)

(73) Assignee: GENOCEA BIOSCIENCES, INC., Cambridge, MA (US)

(21) Appl. No.: 13/140,773

(22) PCT Filed: Dec. 17, 2009
(86) PCT No.: PCT/US09/68457

§ 371 Date: Aug. 17, 2011

Related U.S. Application Data
(62) Division of application No. 61/138,261, filed on Dec. 17, 2008.

(51) Int. Cl. A61K 39/02; A61K 31/711; C07H 21/04

(52) U.S. Cl. 424/263.1; 536/23.7; 536/23.4; 514/44 R

(57) ABSTRACT

Disclosed are novel chlamydia antigens, nucleic acids encoding the antigens, and immunogenic compositions including the antigens. Methods of using the antigens to elicit immune responses (e.g., T cell-mediated and/or B cell-mediated immune responses) comprising administering an immunogenic composition including one or more of the novel antigens may be used for prophylaxis and/or treatment of chlamydia-mediated diseases.

### CROSS REFERENCE TO RELATED APPLICATIONS

The present application is copending with, shares at least one common inventor with and claims priority to U.S. provisional patent application Ser. No. 61/138,261, filed Dec. 17, 2008, the entire contents of which are incorporated herein by reference.

### BACKGROUND OF THE INVENTION

Chlamydia trachomatis is an enormous global public health problem, causing more cases of sexually transmitted disease than any other bacterial pathogen. C. trachomatis is an obligate intracellular bacterium. Infection can cause various disease conditions such as urethritis, cervicitis, pharyngitis, proctitis, epididymitis, and prostatitis. Untreated chlamydial infection can cause pelvic inflammatory disease, which in turn can lead to ectopic pregnancy, infertility, and chronic pelvic pain. Infection during pregnancy has been linked to severe complications such as spontaneous abortion, premature delivery, premature rupture of fetal membranes, low birth weight, and neonatal infections, which are associated with trachoma and pulmonary complications (Navarro et al., Can. J. Inf. Dis. 13(3):195-207, 2002). Trachoma, or conjunctivitis of eyelid and corneal surfaces, is a major disease condition caused by chlamydial infection. Ocular infection is a leading cause of preventable blindness. Pathological effects of C. trachomatis in humans are a significant societal economic burden as well as an ongoing public health concern in both industrialized and developing nations. An estimated four to five million new cases of chlamydial infection occur each year in the United States alone. The annual costs of treating pelvic inflammatory disease may be as high as US $10 billion. The prevalence of C. trachomatis infection in the developing world is over 90%, with an estimated 500 million people at high risk for infection (World Health Organization, Sexually Transmitted Diseases, 2008). There is an urgent need for immunogenic, effective vaccines for controlling chlamydial infections worldwide.

### SUMMARY OF THE INVENTION

The present invention encompasses the discovery of novel antigens from Chlamydia trachomatis that elicit antigen specific immune responses in mammals. Such novel antigens, and/or nucleic acids encoding the antigens, can be incorporated into immunogenic compositions and administered to elicit immune responses, e.g., to provide protection against chlamydia infections and disease caused by chlamydia organisms. Such novel antigens, and/or responses to novel antigens, can be detected to identify and/or characterize immune responses to chlamydia organisms.

Accordingly, in one aspect, the invention provides immunogenic compositions (e.g., vaccines) comprising an isolated chlamydia antigen selected from a CT209 polypeptide antigen, a CT253 polypeptide antigen, a CT425 polypeptide antigen, a CT497 polypeptide antigen, a CT843 polypeptide antigen, and combinations thereof. In some embodiments, a chlamydia antigen comprises a full length chlamydia polypeptide. In some embodiments, a chlamydia antigen comprises a chlamydia polypeptide that lacks a signal sequence and/or transmembrane domain.

In some embodiments, an immunogenic composition comprises a CT209 polypeptide antigen. In some embodiments, a CT209 polypeptide antigen comprises 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, 750, or 800 consecutive amino acids of a CT209 polypeptide sequence. In some embodiments, a CT209 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, 750, or 800 consecutive amino acids of the sequence shown in SEQ ID NO:1.

In some embodiments, an immunogenic composition comprises a CT253 polypeptide antigen. In some embodiments, a CT253 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, or 200 consecutive amino acids of a CT253 polypeptide sequence. In some embodiments, a CT253 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, or 200 consecutive amino acids of the sequence shown in SEQ ID NO:3.

In some embodiments, an immunogenic composition comprises a CT425 polypeptide antigen. In some embodiments, a CT425 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, or 600 consecutive amino acids of a CT425 polypeptide sequence. In some embodiments, a CT425 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, or 600 consecutive amino acids of the sequence shown in SEQ ID NO:5.

In some embodiments, an immunogenic composition comprises a CT497 polypeptide antigen. In some embodiments, a CT497 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, or 450 consecutive amino acids of a CT497 polypeptide sequence. In some embodiments, a CT497 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, or 450 consecutive amino acids of the sequence shown in SEQ ID NO:7.

In some embodiments, an immunogenic composition comprises a CT843 polypeptide antigen. In some embodiments, a CT843 polypeptide antigen comprises at least 8, 9, 10, 11, 12, 13, 14, 15, 16, 17, 18, 19, 20, 25, 30, 35, 45, 50, 60, 65, 70, 75, 80, or 85 consecutive amino acids of a CT843 polypeptide sequence. In some embodiments, a CT843 polypeptide antigen comprises at least 8, 9, 10, 11, 12, 13, 14, 15, 16, 17, 18, 19, 20, 25, 30, 35, 45, 50, 60, 65, 70, 75, 80, or 85 consecutive amino acids of the sequence shown in SEQ ID NO:9.

In some embodiments, an immunogenic composition comprises two or more isolated chlamydia antigens. In some embodiments, the two or more isolated chlamydia antigens comprise two or more of a CT209 polypeptide antigen, a CT253 polypeptide antigen, a CT425 polypeptide antigen, a CT497 polypeptide antigen, and a CT843 polypeptide antigen. In some embodiments, the two or more isolated chlamydia antigens comprise three or more of a CT209 polypeptide antigen, a CT253 polypeptide antigen, a CT425 polypeptide antigen, a CT497 polypeptide antigen, and a CT843 polypeptide antigen. In some embodiments, the two or more isolated chlamydia antigens comprise four or more of a CT209 polypeptide antigen, a CT253 polypeptide antigen, a CT425 polypeptide antigen, a CT497 polypeptide antigen, and a CT843 polypeptide antigen. In some embodiments, the two or more isolated chlamydia antigens comprise a CT209 polypeptide antigen, a CT253 polypeptide antigen, a CT425 polypeptide antigen, a CT497 polypeptide antigen, and a CT843 polypeptide antigen.

In some embodiments, an immunogenic composition comprises two or more isolated chlamydia antigens, wherein the two or more isolated chlamydia antigens comprise (a) a first chlamydia antigen selected from a CT209 polypeptide antigen, a CT253 polypeptide antigen, a CT425 polypeptide antigen, a CT497 polypeptide antigen, and a CT843 polypeptide antigen; and (b) a second chlamydia antigen. In some embodiments, the second chlamydia antigen comprises one or more antigens selected from a CT062 polypeptide antigen, a CT104 polypeptide antigen, a CT144 polypeptide antigen, a CT111 polypeptide antigen, a CT242 polypeptide antigen, a CT491 polypeptide antigen, a CT601 polypeptide antigen, a CT687 polypeptide antigen, a CT732 polypeptide antigen, a CT781 polypeptide antigen, a CT788 polypeptide antigen, a CT808 polypeptide antigen, a CT823 polypeptide antigen, a CT812 polypeptide antigen, a CT681 polypeptide antigen, a CT858 polypeptide antigen, a CT713 polypeptide antigen, an OMP85 polypeptide antigen, a CT315 polypeptide antigen, a CT316 polypeptide antigen, a CT737 polypeptide antigen, and a CT674 polypeptide antigen.

In some embodiments, a chlamydia antigen is fused to a heterologous polypeptide (e.g., an epitope tag).

In some embodiments, an immunogenic composition comprising a chlamydia antigen includes a pharmaceutically acceptable excipient.

In some embodiments, an immunogenic composition comprising a chlamydia antigen includes an adjuvant. In some embodiments, an immunogenic composition includes a mineral-containing adjuvant. In some embodiments, the mineral-containing adjuvant includes aluminum hydroxide. In some embodiments, an immunogenic composition includes an adjuvant comprising an immunomodulatory oligonucleotide. In some embodiments, an immunogenic composition includes an adjuvant comprising an oil emulsion. In some embodiments, an immunogenic composition includes an adjuvant comprising a saponin. In some embodiments, an immunogenic composition includes an adjuvant comprising an immune stimulating complex (ISCOM). In some embodiments, an immunogenic composition includes virus-like particles (VLPs). In some embodiments, an immunogenic composition includes replicons.

In some embodiments, an immunogenic composition elicits an immune response to Chlamydia trachomatis. In some embodiments, an immunogenic composition elicits a T cell mediated immune response to a chlamydia antigen (e.g., a CD4 T cell mediated immune response and/or a CD8 T cell mediated immune response). In some embodiments, an immunogenic composition elicits a Th1 T cell response. In some embodiments, an immunogenic composition elicits an antibody response (e.g., an IgG response, and/or an IgA response).

In another aspect, the invention provides methods for eliciting an immune response against chlamydia in a mammal. The methods include, for example, administering to the mammal an immunogenic composition comprising an isolated chlamydia antigen selected from a CT209 polypeptide antigen, a CT253 polypeptide antigen, a CT425 polypeptide antigen, a CT497 polypeptide antigen, a CT843 polypeptide antigen, and combinations thereof, e.g., an immunogenic composition described herein.

In some embodiments, a method elicits an immune response against Chlamydia trachomatis. In some embodiments, a method elicits a T cell response to a chlamydia antigen (e.g., a CD4 T cell mediated immune response and/or a CD8 T cell mediated immune response). In some embodiments, an immunogenic composition elicits a Th1 T cell response. In some embodiments, a method elicits IFN-γ secretion by antigen specific T cells. In some embodiments, a method elicits an antibody response (e.g., an IgG response, and/or an IgA response).

In some embodiments, a method reduces the incidence of chlamydia infection in subjects administered the composition. In some embodiments, a method reduces the likelihood of lower tract infection by a chlamydia organism. In some embodiments, a method reduces the likelihood of upper tract infection by a chlamydia organism. In some embodiments, a method reduces the likelihood of chronic infection by a chlamydia organism. In some embodiments, a method reduces the likelihood of suffering from pelvic inflammatory disease due to a chlamydia infection.

In some embodiments of a method, an immunogenic composition is administered to the mammal at least two times (e.g., two, three, four, or five times).

In some embodiments, an immunogenic composition administered after a first administration (i.e., as a boost) differs from the composition administered initially, e.g., the composition includes a different chlamydia antigen or a different subset of chlamydia antigens, or a different dose of antigen, or a different adjuvant, or a different dose of adjuvant. In some embodiments, a boost is administered by a different route than a previous administration.

In some embodiments, the mammal is at risk for infection with Chlamydia trachomatis. In some embodiments, the mammal is infected with Chlamydia trachomatis. In some embodiments, the mammal is a female. In some embodiments, the mammal is a human.

In some embodiments, an immunogenic composition administered in a method comprises an adjuvant. In some embodiments, an adjuvant is a mineral-containing adjuvant. In some embodiments, an immunogenic composition administered in a method comprises a pharmaceutically acceptable excipient.

In some embodiments, an immunogenic composition comprises an adjuvant. In some embodiments, an immunogenic composition includes a mineral-containing adjuvant. In some embodiments, a mineral-containing adjuvant includes aluminum hydroxide. In some embodiments, an immunogenic composition includes an adjuvant comprising an immunomodulatory oligonucleotide. In some embodiments, an immunogenic composition includes an adjuvant comprising an oil emulsion. In some embodiments, an immunogenic composition includes an adjuvant comprising a saponin. In some embodiments, an immunogenic composition includes an adjuvant comprising an immune stimulating complex (ISCOM). In some embodiments, an immunogenic composition includes virus-like particles (VLPs). In some embodiments, an immunogenic composition includes replicons.

In some embodiments of provided methods, an immunogenic composition comprises a CT209 polypeptide antigen. In some embodiments, a CT209 polypeptide antigen comprises 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, 750, or 800 consecutive amino acids of a CT209 polypeptide sequence. In some embodiments, a CT209 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, 750, or 800 consecutive amino acids of the sequence shown in SEQ ID NO:1.

In some embodiments of provided methods, an immunogenic composition comprises a CT253 polypeptide antigen. In some embodiments, a CT253 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, or 200 consecutive amino acids of a CT253 polypeptide sequence. In some embodiments, a CT253 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, or 200 consecutive amino acids of the sequence shown in SEQ ID NO:3.

In some embodiments of provided methods, an immunogenic composition comprises a CT425 polypeptide antigen. In some embodiments, a CT425 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, or 600 consecutive amino acids of a CT425 polypeptide sequence. In some embodiments, a CT425 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, or 600 consecutive amino acids of the sequence shown in SEQ ID NO:5.

In some embodiments of provided methods, an immunogenic composition comprises a CT497 polypeptide antigen. In some embodiments, a CT497 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, or 450 consecutive amino acids of a CT497 polypeptide sequence. In some embodiments, a CT497 polypeptide antigen comprises at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, or 450 consecutive amino acids of the sequence shown in SEQ ID NO:7.

In some embodiments of provided methods, an immunogenic composition comprises a CT843 polypeptide antigen. In some embodiments, a CT843 polypeptide antigen comprises at least 8, 9, 10, 11, 12, 13, 14, 15, 16, 17, 18, 19, 20, 25, 30, 35, 45, 50, 60, 65, 70, 75, 80, or 85 consecutive amino acids of a CT843 polypeptide sequence. In some embodiments, a CT843 polypeptide antigen comprises at least 8, 9, 10, 11, 12, 13, 14, 15, 16, 17, 18, 19, 20, 25, 30, 35, 45, 50, 60, 65, 70, 75, 80, or 85 consecutive amino acids of the sequence shown in SEQ ID NO:9.

In some embodiments of provided methods, an immunogenic composition comprises two or more isolated chlamydia antigens. In some embodiments, the two or more isolated chlamydia antigens comprise two or more of a CT209 polypeptide antigen, a CT253 polypeptide antigen, a CT425 polypeptide antigen, a CT497 polypeptide antigen, and a CT843 polypeptide antigen. In some embodiments, the two or more isolated chlamydia antigens comprise three or more of a CT209 polypeptide antigen, a CT253 polypeptide antigen, a CT425 polypeptide antigen, a CT497 polypeptide antigen, and a CT843 polypeptide antigen. In some embodiments, the two or more isolated chlamydia antigens comprise four or more of a CT209 polypeptide antigen, a CT253 polypeptide antigen, a CT425 polypeptide antigen, a CT497 polypeptide antigen, and a CT843 polypeptide antigen. In some embodiments, the two or more isolated chlamydia antigens comprise a CT209 polypeptide antigen, a CT253 polypeptide antigen, a CT425 polypeptide antigen, a CT497 polypeptide antigen, and a CT843 polypeptide antigen.

In some embodiments of provided methods, an immunogenic composition comprises two or more isolated chlamydia antigens, wherein the two or more isolated chlamydia antigens comprise (a) a first chlamydia antigen selected from a CT209 polypeptide antigen, a CT253 polypeptide antigen, a CT425 polypeptide antigen, a CT497 polypeptide antigen, and a CT843 polypeptide antigen; and (b) a second chlamydia antigen. In some embodiments, the second chlamydia comprises one or more antigens selected from a CT062 polypeptide antigen, a CT104 polypeptide antigen, a CT144 polypeptide antigen, a CT111 polypeptide antigen, a CT242 polypeptide antigen, a CT491 polypeptide antigen, a CT601 polypeptide antigen, a CT687 polypeptide antigen, a CT732 polypeptide antigen, a CT781 polypeptide antigen, a CT788 polypeptide antigen, a CT808 polypeptide antigen, a CT823 polypeptide antigen, a CT812 polypeptide antigen, a CT681 polypeptide antigen, a CT858 polypeptide antigen, a CT713 polypeptide antigen, an OMP85 polypeptide antigen, a CT315 polypeptide antigen, a CT316 polypeptide antigen, a CT737 polypeptide antigen, and a CT674 polypeptide antigen.

In some embodiments, an immunogenic composition comprises a chlamydia antigen and an antigen from a different infectious agent. In some embodiments, an immunogenic composition comprises a chlamydia antigen selected from a CT209 polypeptide antigen, a CT253 polypeptide antigen, a CT425 polypeptide antigen, a CT497 polypeptide antigen, a CT843 polypeptide antigen, and combinations thereof; and an antigen from a papillomavirus (e.g., a human papillomavirus). In some embodiments, an immunogenic composition comprises a chlamydia antigen selected from a CT209 polypeptide antigen, a CT253 polypeptide antigen, a CT425 polypeptide antigen, a CT497 polypeptide antigen, a CT843 polypeptide antigen, and combinations thereof; and an antigen from a herpesvirus (e.g., herpes simplex virus-2). In some embodiments, an immunogenic composition comprises a chlamydia antigen selected from a CT209 polypeptide antigen, a CT253 polypeptide antigen, a CT425 polypeptide antigen, a CT497 polypeptide antigen, a CT843 polypeptide antigen, and combinations thereof; and an antigen from N. gonorrhoeae.

In another aspect, the invention provides isolated nucleic acids comprising a nucleotide sequence encoding a chlamydia antigen selected from a CT209 polypeptide antigen, a CT253 polypeptide antigen, a CT425 polypeptide antigen, a CT497 polypeptide antigen, and a CT843 polypeptide antigen. In some embodiments, a nucleic acid further comprises a nucleotide sequence encoding a heterologous peptide fused to the chlamydia antigen.

The invention also provides compositions including nucleic acids encoding a chlamydia antigen as described herein. In some embodiments, a composition includes an isolated nucleic acid comprising a nucleotide sequence encoding a chlamydia antigen selected from a CT209 polypeptide antigen, a CT253 polypeptide antigen, a CT425 polypeptide antigen, a CT497 polypeptide antigen, a CT843 polypeptide antigen, and combinations thereof, and further comprises a pharmaceutically acceptable excipient. In some embodiments, a composition further comprises an adjuvant.

In still another aspect, the invention provides methods for eliciting an immune response against chlamydia in a mammal by administering to the mammal a composition comprising a nucleic acid, wherein the nucleic acid comprises a nucleotide sequence encoding a chlamydia antigen selected from a CT209 polypeptide antigen, a CT253 polypeptide antigen, a CT425 polypeptide antigen, a CT497 polypeptide antigen, a CT843 polypeptide antigen, and combinations thereof.

In another aspect, the invention provides methods for characterizing and/or detecting an immune response to a chlamydia antigen in a subject (e.g., a chlamydia antigen selected from a CT209 polypeptide antigen, a CT253 polypeptide antigen, a CT425 polypeptide antigen, a CT497 polypeptide antigen, a CT843 polypeptide antigen, and combinations thereof). In some embodiments, an immune response in a naïve subject is characterized. In some embodiments, an immune response in a subject infected, or suspected of having been infected, with chlamydia is characterized. In some embodiments, an immune response in a subject administered an immunogenic composition comprising a chlamydia antigen (e.g., an immunogenic composition described herein) is characterized. In some embodiments, an antibody response is characterized. In some embodiments, a T cell response is characterized.

The invention further provides methods of preparing compositions including chlamydia antigens, and antibodies that specifically bind to chlamydia antigens.

Compositions and methods described herein can be used for the prophylaxis and/or treatment of any chlamydial disease, disorder, and/or condition, e.g., any of urethritis, cervicitis, pharyngitis, proctitis, epididymitis, prostatitis, pelvic inflammatory disease, and trachoma, due to a chlamydia infection. In some embodiments, an immunogenic composition described herein reduces risk of infection by, and/or treats, alleviates, ameliorates, relieves, delays onset of, inhibit progression of, reduces severity of, and/or reduce incidence of one or more symptoms or features of a chlamydial disease, disorder, and/or condition. In some embodiments, the prophylaxis and/or treatment of chlamydia infection comprises administering a therapeutically effective amount of an immunogenic composition comprising a novel chlamydial antigen described herein to a subject in need thereof, in such amounts and for such time as is necessary to achieve the desired result. In certain embodiments of the present invention a “therapeutically effective amount” of an inventive immunogenic composition is that amount effective for treating, alleviating, ameliorating, relieving, delaying onset of, inhibiting progression of, reducing severity of, and/or reducing incidence of one or more symptoms or features of chlamydia infection.

In some embodiments, inventive prophylactic and/or therapeutic protocols involve administering a therapeutically effective amount of one or more immunogenic compositions comprising a novel chlamydia antigen to a subject such that an immune response is stimulated in one or both of T cells and B cells.

The present invention provides novel immunogenic compositions comprising a therapeutically effective amount of one or more chlamydia antigens (e.g., one or more of a CT209 polypeptide antigen, a CT253 polypeptide antigen, a CT425 polypeptide antigen, a CT497 polypeptide antigen, a CT843 polypeptide antigen, and combinations thereof) and one or more pharmaceutically acceptable excipients. In some embodiments, the present invention provides for pharmaceutical compositions comprising an immunogenic composition as described herein. In accordance with some embodiments, a method of administering a pharmaceutical composition comprising inventive compositions to a subject (e.g. human, e.g., a child, adolescent, or young adult) in need thereof is provided.

In some embodiments, a therapeutically effective amount of an immunogenic composition is delivered to a patient and/or animal prior to, simultaneously with, and/or after diagnosis with a chlamydial disease, disorder, and/or condition. In some embodiments, a therapeutic amount of an inventive immunogenic composition is delivered to a patient and/or animal prior to, simultaneously with, and/or after onset of symptoms of a chlamydial disease, disorder, and/or condition.

In some embodiments, immunogenic compositions of the present invention are administered by any of a variety of routes, including oral, intramuscular, subcutaneous, transdermal, interdermal, rectal, intravaginal, mucosal, nasal, buccal, enteral, sublingual; by intratracheal instillation, bronchial instillation, and/or inhalation; and/or as an oral spray, nasal spray, and/or aerosol. In some embodiments, immunogenic compositions of the present invention are administered by a variety of routes, including intravenous, intra-arterial, intramedullary, intrathecal, intraventricular, transdermal, intraperitoneal, topical (as by powders, ointments, creams, and/or drops), transdermal, or by intratracheal instillation.

In certain embodiments, an immunogenic composition may be administered in combination with one or more additional therapeutic agents which treat the symptoms of chlamydia infection (e.g., with an antibiotic such as an erythromycin or a tetracyclin).

The invention provides a variety of kits comprising one or more of the immunogenic compositions of the invention. For example, the invention provides a kit comprising an immunogenic composition comprising a chlamydia antigen, or a nucleic acid encoding the antigen, wherein the antigen is selected from a CT209 polypeptide antigen, a CT253 polypeptide antigen, a CT425 polypeptide antigen, a CT497 polypeptide antigen, a CT843 polypeptide antigen, and combinations thereof; and instructions for use. A kit may comprise multiple different chlamydia antigens. A kit may comprise any of a number of additional components or reagents in any combination. According to certain embodiments of the invention, a kit may include, for example, (i) a chlamydia antigen selected from a CT209 polypeptide antigen, a CT253 polypeptide antigen, a CT425 polypeptide antigen, a CT497 polypeptide antigen, a CT843 polypeptide antigen, and combinations thereof; (ii) an adjuvant; and (iii) instructions for administering a composition including the chlamydia antigen and the adjuvant to a subject in need thereof.

This application refers to various issued patents, published patent applications, journal articles, database entries containing amino acid and nucleic acid sequence information, and other publications, all of which are incorporated herein by reference.

### BRIEF DESCRIPTION OF THE DRAWING

FIG. 1 is a graph showing results of a representative C. trachomatis ORFeome library screen. Peritoneal CD8 T cells isolated and expanded from C57BL/6 mice that had been infected six days prior with 5×10⁵ IFU of Chlamydia trachomatis were exposed to antigen presenting cells that had been pulsed with clones from the proteomic library and subsequently fixed. The graph shows IFNγ cytokine concentrations from supernatants harvested after 24 hours as determined by ELISA. Each data point represents a single clone in the library, and the line indicates the cutoff of the mean plus 2×SD. Control samples of mitogen-stimulated T cells (triangle) and T cells cultured with E. coli expressing irrelevant antigens (squares) are also indicated.

FIG. 2 is a graph showing the frequency of T cell responses to antigens from Chlamydia trachomatis in multiple animal strains. T cell responses to C. trachomatis antigens were evaluated in C57BL/6, Balb/c, CD1, and C3H mice. The graph represents the frequency with which each protein was identified across all strains. Each symbol represents a single clone in the library. These data are inclusive of nine screens of either CD4 or CD8 T cells.

### DEFINITIONS

Adjuvant: As used herein, the term “adjuvant” refers to an agent that alters (e.g., enhances) an immune response to an antigen. In some embodiments, an adjuvant is used to enhance an immune response to a peptide antigen administered to a subject. In some embodiments, an adjuvant is used to enhance an immune response to an antigen encoded by a nucleic acid administered to a subject.

Antibody: As used herein, the term “antibody” refers to any immunoglobulin, whether natural or wholly or partially synthetically produced. All derivatives thereof which maintain specific binding ability are also included in the term. The term also covers any protein having a binding domain which is homologous or largely homologous to an immunoglobulin binding domain. Such proteins may be derived from natural sources, or partly or wholly synthetically produced. An antibody may be monoclonal or polyclonal. An antibody may be a member of any immunoglobulin class, including any of the human classes: IgG, IgM, IgA, IgD, and IgE. As used herein, the terms “antibody fragment” or “characteristic portion of an antibody” are used interchangeably and refer to any derivative of an antibody which is less than full-length. In general, an antibody fragment retains at least a significant portion of the full-length antibody's specific binding ability. Examples of antibody fragments include, but are not limited to, Fab, Fab′, F(ab′)2, scFv, Fv, dsFv diabody, and Fd fragments. An antibody fragment may be produced by any means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains which are linked together, for example, by disulfide linkages. An antibody fragment may optionally comprise a multimolecular complex. A functional antibody fragment will typically comprise at least about 50 amino acids and more typically will comprise at least about 200 amino acids.

Antigen: The term “antigen”, as used herein, refers to a molecule (e.g., a polypeptide) that elicits a specific immune response. Antigen specific immunological responses, also known as adaptive immune responses, are mediated by lymphocytes (e.g., T cells, B cells) that express antigen receptors (e.g., T cell receptors, B cell receptors). In certain embodiments, an antigen is a T cell antigen, and elicits a cellular immune response. In certain embodiments, an antigen is a B cell antigen, and elicits a humoral (i.e., antibody) response. In certain embodiments, an antigen is both a T cell antigen and a B cell antigen. As used herein, the term “antigen” encompasses both a full length polypeptide as well as a portion of the polypeptide, that represent immunogenic fragments (i.e., fragments that elicit an antigen specific T cell response, B cell response, or both) of such complete polypeptides. In some embodiments, antigen is a peptide epitope found within a polypeptide sequence (e.g., a peptide epitope bound by a Major Histocompatibility Complex (MHC) molecule (e.g., MHC class I, or MHC class II). Accordingly, peptides 5-15 amino acids in length, and longer polypeptides, e.g., having 60, 70, 75, 80, 85, 90, 100, 150, 200 250, or more amino acids, can be “antigens”. In one example, the present invention provides a CT209 polypeptide antigen. In some embodiments, a CT209 polypeptide antigen includes a full length CT209 polypeptide amino acid sequence (e.g., a full length CT209 polypeptide of SEQ ID NO:1). In some embodiments, a CT209 antigen polypeptide includes a portion of a CT209 polypeptide (e.g., a portion of the CT209 polypeptide of SEQ ID NO:1, e.g., which portion includes at least 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, or 800 contiguous amino acids of a sequence at least 80%, 85%, 90%, 95%, 97%, 98%, or 99% identical to SEQ ID NO:1). In some embodiments, a CT209 antigen polypeptide includes a portion of a CT209 polypeptide (e.g., a portion of the CT209 polypeptide of SEQ ID NO:1, e.g., which portion includes at least 8, 9, 10, 11, 12, 13, 14, 15, 16, 17, 18, 19, 20, 25, 30, 35, 40, 45, 50, 55, 60, 65, 70, 75, 80, 85, 90, 95, 100, 150, 200, 250, 300, 350, 400, 450, 500, 550, 600, 650, 700, 750, or 800 contiguous amino acids of SEQ ID NO:1).

Approximately: As used herein, the terms “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

Chlamydia antigen: As used herein, the term “chlamydia antigen” refers to an antigen that elicits an antigen specific immune response against any organism of the Chlamydia genus, such as a Chlamydia trachomatis organism, a Chlamydia psittaci organism, or a Chlamydia pneumoniae organism, a Chlamydia suis organism, a Chlamydia muridarum organism, etc. In some embodiments, a chlamydia antigen elicits an antigen specific immune response against chlamydia organisms of multiple species (e.g., two or three of Chlamydia trachomatis, Chlamydia psittaci, and Chlamydia pneumoniae). For example, a C. trachomatis CT209 polypeptide has a very high degree of identity to a CT843 polypeptide of C. pneumoniae. Thus, in some embodiments, a C. trachomatis CT209 polypeptide elicits an antigen specific immune response against both C. trachomatis CT209 and C. pneumoniae CT843 polypeptides. CT253 polypeptides of C. trachomatis and C. pneumoniae also exhibit a high degree of identity and, in some embodiments, a CT253 polypeptide of one species can elicit antigen specific immune responses to both. In some embodiments, a chlamydia antigen elicits an antigen specific immune response against chlamydia organisms of multiple serovars (e.g., one or more of serovars A, B, Ba, C, D, E, F, G, H, I, J, K, L1, L2, L3 of C. trachomatis). Chlamydia antigens include full length polypeptides encoded by chlamydia genes, as well as immunogenic portions of the polypeptides.

Immunogenic composition: As used herein, the term “immunogenic composition” refers to a composition that includes a molecule that induces an immune response in a subject. In some embodiments, an immunogenic composition includes a polypeptide or peptide antigen. In some embodiments, an immunogenic composition includes a nucleic acid encoding a polypeptide or peptide antigen. An immunogenic composition can include molecules that induce an immune response against multiple antigens.

In vitro: As used herein, the term “in vitro” refers to events that occur in an artificial environment, e.g., in a test tube or reaction vessel, in cell culture, etc., rather than within an organism (e.g., animal, plant, and/or microbe).

In vivo: As used herein, the term “in vivo” refers to events that occur within an organism (e.g., animal, plant, and/or microbe).

Isolated: The term “isolated”, as used herein, means that the isolated entity has been separated from at least one component with which it was previously associated. When most other components have been removed, the isolated entity is “purified.” Isolation and/or purification and/or concentration may be performed using any techniques known in the art including, for example, chromatography, fractionation, precipitation, or other separation.

Nucleic acid: As used herein, the term “nucleic acid,” in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As used herein, the terms “oligonucleotide” and “polynucleotide” can be used interchangeably. In some embodiments, “nucleic acid” encompasses RNA as well as single and/or double-stranded DNA and/or cDNA. Furthermore, the terms “nucleic acid,” “DNA,” “RNA,” and/or similar terms include nucleic acid analogs, i.e. analogs having other than a phosphodiester backbone. The term “nucleotide sequence encoding an amino acid sequence” includes all nucleotide sequences that are degenerate versions of each other and/or encode the same amino acid sequence. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, backbone modifications, etc. A nucleic acid sequence is presented in the 5′ to 3′ direction unless otherwise indicated.

Polypeptide: The term “polypeptide”, as used herein, generally has its art-recognized meaning of a polymer of at least three amino acids. However, the term is also used to refer to specific classes of antigen polypeptides, such as, for example, CT209 polypeptides, CT253 polypeptides, CT425 polypeptides, CT497 polypeptides, and CT843 polypeptides. For each such class, the present specification provides several examples of known sequences of such polypeptides. Those of ordinary skill in the art will appreciate, however, that the term “polypeptide”, as used herein to refer to “polypeptide antigen”, is intended to be sufficiently general as to encompass not only polypeptides having a sequence recited herein, but also to encompass polypeptides having a variation of the sequence that elicits an antigen-specific response to the polypeptide. For example, a “CT209 polypeptide” includes the CT209 polypeptide shown in SEQ ID NO:1, as well as polypeptides that have variations of a SEQ ID NO:1 sequence and that maintain the ability to elicit an antigen-specific response to a polypeptide of SEQ ID NO:1. Those of ordinary skill in the art understand that protein sequences generally tolerate some substitution without destroying immunogenicity and antigen specificity. Thus, any polypeptide that retains immunogenicity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented herein.

One example of an algorithm that is suitable for determining percent sequence identity and sequence similarity is the BLAST algorithm, which is described in Altschul et al., Nuc. Acids Res. 25:3389-3402, 1977. BLAST is used, with the parameters described herein, to determine percent sequence identity for the nucleic acids and proteins of the present disclosure. Software for performing BLAST analysis is publicly available through the National Center for Biotechnology Information (available at the following internet address: ncbi.nlm.nih.gov). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA, 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA, 90:5873-5787, 1993). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

Subject: As used herein, the term “subject” or “patient” refers to any organism to which a composition of this invention may be administered, e.g., for experimental, diagnostic, and/or therapeutic purposes. Typical subjects include mammals such as mice, rats, rabbits, non-human primates, and humans.

Suffering from: An individual who is “suffering from” a disease, disorder, and/or condition has been diagnosed with or displays one or more symptoms of the disease, disorder, and/or condition.

Susceptible to: An individual who is “susceptible to” a disease, disorder, and/or condition has not been diagnosed with and/or may not exhibit symptoms of the disease, disorder, and/or condition. In some embodiments, a disease, disorder, and/or condition is associated with a chlamydia infection (e.g., a C. trachomatis infection, a C. pneumoniae infection, or a C. psittaci infection). In some embodiments, an individual who is susceptible to a chlamydia infection may be exposed to a chlamydia microbe (e.g., by ingestion, inhalation, physical contact, etc.). In some embodiments, an individual who is susceptible to a chlamydia infection may be exposed to an individual who is infected with the microbe. In some embodiments, an individual who is susceptible to a chlamydia infection is one who is in a location where the microbe is prevalent (e.g., one who is traveling to a location where the microbe is prevalent). In some embodiments, an individual who is susceptible to a chlamydia infection is susceptible due to young age (e.g., a child, adolescent, or young adult). In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutically effective amount: As used herein, the term “therapeutically effective amount” means an amount of a therapeutic, prophylactic, and/or diagnostic agent (e.g., inventive immunogenic composition) that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

Therapeutic agent: As used herein, the phrase “therapeutic agent” refers to any agent that, when administered to a subject, has a therapeutic, prophylactic, and/or diagnostic effect and/or elicits a desired biological and/or pharmacological effect.

Treating: As used herein, the term “treating” refers to partially or completely alleviating, ameliorating, relieving, delaying onset of, inhibiting progression of, reducing severity of, and/or reducing incidence of one or more symptoms or features of a particular disease, disorder, and/or condition. For example, “treating” a microbial infection may refer to inhibiting survival, growth, and/or spread of the microbe. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. In some embodiments, treatment comprises delivery of an immunogenic composition (e.g., a vaccine) to a subject.

Vaccine: As used herein, the term “vaccine” refers to an entity comprising at least one immunogenic component (e.g., an immunogenic component which includes a peptide or protein, and/or an immunogenic component which includes a nucleic acid). In certain embodiments, a vaccine includes at least two immunogenic components. In some embodiments, a vaccine is capable of stimulating an immune response of both T cells and B cells. In some embodiments, any assay available in the art may be used to determine whether T cells and/or B cells have been stimulated. In some embodiments, T cell stimulation may be assayed by monitoring antigen-induced production of cytokines, antigen-induced proliferation of T cells, and/or antigen-induced changes in protein expression. In some embodiments, B cell stimulation may be assayed by monitoring antibody titers, antibody affinities, antibody performance in neutralization assays, class-switch recombination, affinity maturation of antigen-specific antibodies, development of memory B cells, development of long-lived plasma cells that can produce large amounts of high-affinity antibodies for extended periods of time, germinal center reactions, and/or antibody performance in neutralization assays. In some embodiments, a vaccine further includes at least one adjuvant that can help stimulate an immune response in T cells and/or B cells.

### DETAILED DESCRIPTION OF CERTAIN PREFERRED EMBODIMENTS OF THE INVENTION

Infection by Chlamydia trachomatis causes inflammation and damage to mucosal tissues, leading to pathologies such as urethritis, cervicitis, pharyngitis, proctitis, epididymitis, prostatitis, and trachoma. Chlamydia bacteria, which primarily infect epithelial cells, alternate between two developmental forms, the elementary body (EB) and reticulate body (RB). EB forms of chlamydia are infectious and invade host cells. After forming an inclusion within host cells, EB forms differentiate into RB forms which replicate for a period of time and differentiate back to EB forms. C. trachomatis species are categorized into serovars based on reactivity of patient sera to the major outer membrane protein (MOMP). Serovars A, B, Ba, and C are associated with infection of conjunctival epithelium. Servovars D-K are associated with urogenital tract infections. Serovars L1-L3 are associated with urogenital tract infection and a systemic condition, lymphogranuloma venereum.

Various arms of the adaptive immune system appear to play a role in responding to chlamydial infections. CD4 T cell responses of the Th1 subtype have been shown to be important for clearance of chlamydia infections in an animal model (Morrison et al., Infect. Immun. 70:2741-2751, 2002). B cell responses are thought to contribute to protective immunity in humans and non-human primates (Brunham et al., Infect. Immun. 39:1491-1494, 1983; Taylor et al., Invest. Ophthalmol. Vis. Sci 29:1847-1853, 1988). CD8 T cells have lytic functions that are important for the control of intracellular pathogens. Chlamydia-specific CD8 T cells have been isolated from infected humans, indicating a role for these cells in responding to chlamydia infections (Gervassi et al., J. Immunol. 171: 4278-4286, 2003).

The present invention provides chlamydia antigens, including CT209 polypeptide antigens, CT253 polypeptide antigens, CT425 polypeptide antigens, CT497 polypeptide antigens, CT843 polypeptide antigens, that are recognized by immune cells (e.g., T cells) of infected mammals. As described in the Examples herein, these antigens were discovered as targets of cellular immunity in vivo. Accordingly, these antigens provide novel compositions for eliciting immune responses with the aim of eliciting beneficial immune responses, e.g., to protect against chlamydia infections and associated pathologies. These antigens also provide novel targets for characterizing chlamydia infections and immune responses to chlamydia infections.

CT209 polypeptides are thought to function as cytoplasmic leucyl-tRNA synthetases in chlamydia organisms. Exemplary amino acid and nucleotide sequences from a full length CT209 polypeptide of C. trachomatis are shown below in Table 1. In some embodiments, a CT209 polypeptide antigen includes at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, 750, or 800 consecutive amino acids of a CT209 polypeptide sequence, e.g., at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, 600, 650, 700, 750, or 800 consecutive amino acids of the sequence shown in SEQ ID NO:1. In some embodiments, a CT209 polypeptide antigen is a full length CT209 polypeptide (e.g., the antigen comprises the amino acid sequence of SEQ ID NO:1).

Exemplary amino acid and nucleotide sequences from a full length CT253 polypeptide of C. trachomatis are shown in Table 1. In some embodiments, a CT253 polypeptide antigen includes at least 8, 9, 10, 11, 12, 13, 14, 15, 16, 17, 18, 19, 20, 25, 30, 35, 45, 50, 60, 65, 70, 75, 80, 85, 90, 95, 100, 150, or 200 consecutive amino acids of a CT253 polypeptide sequence, e.g., at least 8, 9, 10, 11, 12, 13, 14, 15, 16, 17, 18, 19, 20, 25, 30, 35, 45, 50, 60, 65, 70, 75, 80, 85, 90, 95, 100, 150, or 200 consecutive amino acids of the sequence shown in SEQ ID NO:3. In some embodiments, a CT253 polypeptide antigen lacks a transmembrane domain and/or signal sequence (e.g., a CT253 polypeptide antigen lacks amino acids 1-24 of SEQ ID NO:3). In some embodiments, a CT253 polypeptide antigen is a full length CT253 polypeptide (e.g., the antigen comprises the amino acid sequence of SEQ ID NO:3)

Exemplary amino acid and nucleotide sequences from a full length CT425 polypeptide of C. trachomatis are shown in Table 1. In some embodiments, a CT425 polypeptide antigen includes at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, or 600 consecutive amino acids of a CT425 polypeptide sequence, e.g., at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, 450, 500, 550, or 600 consecutive amino acids of the sequence shown in SEQ ID NO:5. In some embodiments, a CT425 polypeptide antigen is a full length CT425 polypeptide (e.g., the antigen comprises the amino acid sequence of SEQ ID NO:5).

CT497 polypeptides are replicative DNA helicases. Exemplary amino acid and nucleotide sequences from a full length CT497 polypeptide of C. trachomatis are shown below in Table 1. In some embodiments, a CT497 polypeptide antigen includes at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, or 450 consecutive amino acids of a CT497 polypeptide sequence, e.g., at least 8, 9, 10, 11, 12, 13, 14, 15, 16, 17, 18, 19, 20, 25, 30, 35, 45, 50, 60, 65, 70, 75, 80, 85, 90, 95, 100, 150, 200, 250, 300, 350, 400, or 450 consecutive amino acids of the sequence shown in SEQ ID NO:7. In some embodiments, a CT497 polypeptide antigen lacks a transmembrane domain (e.g., a CT497 polypeptide antigen lacks amino acids 25-44 of SEQ ID NO:7). In some embodiments, a CT497 polypeptide antigen is a full length CT497 polypeptide (e.g., the antigen comprises the amino acid sequence of SEQ ID NO:7).

CT843 polypeptides are 30S ribosomal S15 proteins. Exemplary amino acid and nucleotide sequences from a full length CT843 polypeptide of C. trachomatis are shown below in Table 1. In some embodiments, a CT843 polypeptide antigen includes at least 8, 9, 10, 11, 12, 13, 14, 15, 16, 17, 18, 19, 20, 25, 30, 35, 45, 50, 60, 65, 70, 75, 80, or 85 consecutive amino acids of a CT843 polypeptide sequence, e.g., at least 8, 9, 10, 11, 12, 13, 14, 15, 16, 17, 18, 19, 20, 25, 30, 35, 45, 50, 60, 65, 70, 75, 80, or 85 consecutive amino acids of the sequence shown in SEQ ID NO:9. In some embodiments, a CT843 polypeptide antigen is a full length CT843 polypeptide (e.g., the antigen comprises the amino acid sequence of SEQ ID NO:9).

TABLE 1
Exemplary Chlamydia Polypeptide Amino Acid and Nucleotide Sequences

| Name | |
|---|---|
| Genbank GI No./ | |
| GenBank Acc. No. | |
| or position in | |
| genomic sequence | |
| under Acc. No. | |
| NC_000117.1 | |
| GI:15604717 | Nucleotide/Amino Acid Sequence (SEQ ID NO:) |
| CT209 | MRYDPGLIEEKWQKFWENEQVFKAEEDETKTKYYVLDMFPYPSGAGLHVGHLIGYTATDI |
| leucyl-tRNA | VARYKRAQGFSVLHPMGWDSFGLPAEQYAIRTGTHPRETTEKNIANFKKQLTAMGFSYDE |
| synthetase | SREFATSDPEYYKWTQKLFLILYEKGLAYMADMAVNYCPELGTVLSNEEIENGFSVDGGY |
| gi\|15604929\|ref | PVERRMLRQWVLRITAFADQLLEGLDELDWPESVKQLQKNWIGKSSGASVNFATEHGVIE |
| \|NP_219713.1\| | VFTTRPDTLIGVSFLALAPEHPLVDLLTSDEQKAVVAQYIKETQSKSERDRISEMKTKSG |
| | VFTGSYAKHPVTHKLIPIWIADYVLIGFGSGAVMGVPAHDERDLLFAEQFNLPVVSVLNK |
| | EGVCINSCCEGFHLDGLSGEEAKQYVINFLEENHLGAAKIAYKLRDWLFSRQRYWGEPIP |
| | IIHFEDGSCRPLRDYELPLLPPEIQDYRPEGVGQGPLAKVREWVQVFDTETQRAGKRETH |
| | TMPQWAGSCWYYLRFCDAHNSAAPWAKEKEQYWMPVDLYIGGAEHAVLHLLYARFWHQVF |
| | YEAGIVSTPEPFKKLVNQGLVLATSYRIPGKGYIYPEIAKEENGKWVAPSGEELDVRQEK |
| | MSKSKLNGVDPQILIDEFGADAVRMYAMFSGPLDKNKLWSNQGVAGCRRFLNRFYEMVSS |
| | DRVKEDNNFEGLSLAHKLVQRVTDAIEKLSLNTIPSSFMEFINDFVKLAVYPKSAVEMAV |
| | RALAPIAPHISEELWVLLGNSPGVQKSGWPSVLPEYLEGQTVTIVVQVNGKLRARLDIMK |
| | DASKEEVLALARESASKYLEGCEVKKAIFVPARLVNFVV (SEQ ID NO: 1) |
| CT209 | ATGCGCTATGATCCCGGTCTCATAGAAGAAAAATGGCAAAAGTTTTGGGAGAACGAGCAA |
| gi\|15604717: | GTTTTTAAGGCTGAGGAGGATGAAACTAAAACAAAATACTATGTTTTAGATATGTTTCCT |
| 235766-238225 | TATCCATCTGGGGCAGGATTGCACGTGGGGCATCTAATTGGCTATACAGCAACAGATATT |
| Chlamydia | GTCGCAAGGTATAAGCGAGCCCAGGGGTTTTCAGTTTTACATCCTATGGGTTGGGATAGT |
| trachomatis | TTTGGGTTGCCTGCAGAGCAGTATGCTATACGCACGGGGACACACCCAAGAGAGACCACA |
| D/UW-3/CX, | GAGAAAAACATCGCTAATTTCAAGAAACAGTTAACTGCAATGGGGTTCTCTTACGACGAA |
| complete genome | AGTCGAGAGTTCGCTACTTCAGATCCAGAATATTATAAATGGACTCAAAAGCTTTTTTTA |
| | ATCCTTTATGAGAAAGGATTAGCCTATATGGCCGATATGGCTGTTAACTATTGTCCTGAG |
| | TTGGGGACTGTTTTATCGAATGAAGAGATTGAAAACGGATTTTCTGTTGATGGGGGGTAT |
| | CCTGTTGAAAGGAGAATGCTTCGCCAATGGGTGCTGCGTATTACAGCATTCGCGGATCAG |
| | CTCCTAGAAGGACTAGATGAGCTGGATTGGCCAGAAAGTGTCAAACAATTGCAGAAGAAT |
| | TGGATTGGGAAGTCTTCAGGAGCTTCTGTTAATTTTGCCACAGAACATGGAGTAATAGAA |
| | GTTTTCACAACAAGACCGGACACTTTGATAGGAGTTTCTTTTTTAGCCTTAGCTCCTGAA |
| | CATCCCTTGGTCGATCTCCTCACTTCTGATGAGCAAAAGGCTGTTGTGGCACAGTATATT |
| | AAAGAAACACAAAGTAAAAGCGAAAGAGATCGCATCAGCGAGATGAAAACTAAGAGCGGG |
| | GTCTTTACGGGATCTTACGCAAAACATCCTGTCACTCACAAACTTATCCCTATCTGGATC |
| | GCAGATTATGTATTAATAGGCTTTGGTTCAGGAGCTGTCATGGGAGTTCCTGCTCATGAC |
| | GAACGTGATTTGCTTTTTGCCGAACAGTTTAATTTACCTGTAGTCTCCGTGCTTAATAAA |
| | GAAGGTGTCTGTATTAATAGCTGTTGTGAAGGGTTCCATTTGGATGGATTGTCCGGAGAA |
| | GAAGCTAAGCAATATGTGATTAACTTTTTAGAAGAGAATCATCTTGGAGCAGCAAAAATC |
| | GCTTATAAGTTGCGAGACTGGCTATTCTCGCGTCAACGTTATTGGGGGGAGCCTATACCG |
| | ATTATCCATTTTGAAGATGGCTCTTGTCGTCCTTTAAGAGACTATGAACTTCCATTGTTA |
| | CCTCCGGAAATTCAAGATTATCGACCAGAAGGGGTCGGACAAGGACCTTTGGCTAAGGTG |
| | CGAGAGTGGGTACAAGTTTTCGACACGGAGACACAACGAGCTGGGAAACGTGAAACACAT |
| | ACCATGCCTCAATGGGCAGGCTCTTGCTGGTACTATTTACGTTTTTGCGATGCCCACAAC |
| | TCGGCCGCTCCCTGGGCAAAGGAAAAGGAACAATATTGGATGCCAGTAGATCTGTATATT |
| | GGAGGTGCAGAACATGCTGTACTGCACTTACTTTATGCGCGTTTCTGGCATCAGGTTTTC |
| | TATGAAGCTGGGATTGTTTCAACACCCGAGCCTTTTAAGAAGCTTGTCAATCAAGGCTTG |
| | GTATTGGCGACATCTTATCGCATTCCTGGGAAAGGATATATATATCCTGAAATAGCAAAA |
| | GAAGAAAACGGGAAATGGGTTGCTCCTTCGGGAGAAGAGTTGGATGTCCGTCAAGAAAAG |
| | ATGTCGAAATCCAAATTAAATGGGGTGGATCCTCAGATCTTGATAGATGAATTTGGAGCT |
| | GACGCTGTTCGGATGTACGCAATGTTTTCAGGGCCTTTAGATAAAAATAAACTCTGGTCT |
| | AACCAAGGAGTTGCTGGTTGCAGACGATTTTTGAATCGTTTTTATGAAATGGTCTCTTCT |
| | GATCGAGTTAAGGAAGATAACAATTTTGAAGGCTTATCATTAGCGCATAAGCTTGTGCAG |
| | CGAGTAACAGATGCTATTGAAAAATTGTCTTTGAATACGATACCGTCCTCTTTTATGGAG |
| | TTTATCAATGACTTTGTGAAGCTCGCTGTATATCCGAAGAGTGCTGTAGAAATGGCTGTT |
| | CGGGCTTTGGCTCCAATTGCCCCTCACATTAGTGAAGAGTTGTGGGTGTTGTTAGGCAAT |
| | TCTCCTGGTGTTCAGAAGTCCGGATGGCCGAGTGTTTTGCCTGAGTATTTAGAAGGACAG |
| | ACAGTTACTATTGTAGTTCAGGTAAATGGTAAACTTCGAGCTCGACTAGATATTATGAAG |
| | GATGCTTCTAAAGAAGAAGTCCTAGCGTTAGCAAGGGAATCTGCATCTAAGTACTTAGAG |
| | GGGTGTGAAGTGAAAAAGGCTATTTTTGTTCCTGCTCGATTGGTAAACTTTGTTGTATGA |
| | (SEQ ID NO: 2) |
| CT253 | MRKFWLLASFGLLSLTTTTLSSCAVSNSGSYNARLYTKGSKAKGVVAMLPVFYRTEKSAE |
| gi\|15604974\|ref | LLPWNLQAEFSEEISRRLHSSDKLLLIKHHASAGVAAQFFSPTPNISPELATQLLPAEFV |
| \|NP_219758.1\| | VAAEILEQKTTEDVLNPSISASVRVRVFDIRHNKVSMIYQEILDASQSLASGSNDYHRYG |
| | WRSKNFDSTPMGLMHQRLFREIVARVEGYVCANYS (SEQ ID NO: 3) |
| CT253 | ATGCGAAAATTCTGGTTACTTGCTTCTTTCGGCCTTTTGTCTTTAACTACGACTACTCTT |
| gi\|15604717: | TCTAGCTGTGCTGTATCTAATTCTGGCAGCTACAATGCTAGACTATACACTAAAGGGAGC |
| 285078-285725 | AAGGCTAAAGGAGTCGTCGCCATGCTACCTGTTTTTTATCGAACAGAAAAGTCTGCAGAA |
| Chlamydia | CTACTCCCTTGGAATTTACAAGCAGAGTTTTCCGAAGAGATTAGCAGACGTTTGCACTCT |
| trachomatis | TCTGATAAACTACTTTTAATCAAACACCACGCTTCAGCTGGTGTTGCTGCACAATTTTTT |
| D/UW-3/CX, | TCTCCTACTCCTAATATTTCGCCCGAATTAGCGACTCAGCTGTTGCCTGCGGAATTCGTA |
| complete genome | GTTGCGGCAGAAATTTTAGAACAGAAAACAACGGAAGATGTTTTAAACCCTTCTATTTCA |
| | GCATCTGTTCGTGTGCGAGTGTTTGATATTCGTCACAATAAAGTCTCTATGATATACCAA |
| | GAGATTTTAGACGCGAGTCAATCTCTCGCCTCTGGAAGCAACGATTATCATCGTTATGGC |
| | TGGCGTTCGAAAAACTTCGATTCGACTCCGATGGGCCTCATGCATCAGAGATTATTTAGA |
| | GAGATTGTTGCTCGCGTAGAGGGATATGTCTGCGCAAACTACTCTTGA (SEQ ID |
| | NO: 4) |
| CT425 | MRRSVCYVTPSVARAGQISTWRFEYSSANFLPEGTLLKFDLGIDGRPIDWEIPSTDLSQP |
| gi\|15605152\|ref | CNTIYLETPSEDIVAAKAVYAPGGYIPTFEFTLPCDVEAGDTFSIILGSSPNFPQEDSSG |
| \|NP_219937.1\| | NGAQLFTQRRKPFSLYVDPSGKGSFEDPDIFTMDIRGNVLKNIRIFAPSYVIKNKRFDIT |
| | VRFEDEFGNLTNFSPEETHIELSYEHLRENLNWQLFIPETGFVILPNLYFNEPGIYRIQL |
| | RNQATKEVFTSAPIKCFAETSSHLLWGLLHGESERVDSEGNIESCLRYFRDDCALNFFAT |
| | SSFEIQDGLTPETIKTINQTVADFNEEDRFIALSGAQYLSEEPGEGIREVLLMKEPKSPG |
| | KHKECKLFPLSKLYKQSTSHELISIPSFTASKKFGYNFNNFHPEFERVVEIYNAWGCSER |
| | TEAEGNPFPIKGSIDSENPEGTVLSALKRNLRFGFVAGGLDDRNLYNHFFDSDQQQYSPG |
| | LTAVICNKYSRDSLLEALYQRQCYATTGQRIIVNFQITSAPMGSELSTAIKPGLVINRHI |
| | SGYVAGTAKIASIEIIRNEDILHTFHPDGNNFEYEYDDLSPFAQVTLKDPQNGAPFAFYY |
| | LRVTQENGAMAWSSPIWIDLN (SEQ ID NO: 5) |
| CT425 | ATGCGCAGATCTGTTTGTTACGTTACTCCTTCAGTTGCTAGGGCTGGTCAAATTTCTACC |
| gi\|15604717: | TGGCGTTTCGAATATTCTTCAGCTAATTTCCTTCCCGAAGGCACATTGCTAAAATTTGAC |
| 493898-495763 | CTGGGAATAGACGGACGCCCTATAGACTGGGAGATTCCTTCTACAGATCTTTCTCAACCA |
| | TGTAATACAATTTATTTAGAAACGCCTTCCGAGGATATTGTGGCTGCAAAAGCTGTGTAT |
| | GCTCCCGGAGGCTATATCCCTACTTTCGAATTTACTCTCCCTTGTGATGTGGAAGCTGGG |
| | GACACTTTCTCTATTATTCTTGGCTCCTCTCCCAACTTCCCTCAAGAGGACTCTTCAGGT |
| | AATGGTGCTCAATTATTTACTCAACGCCGTAAACCTTTCTCTCTTTATGTTGACCCATCA |
| | GGGAAAGGATCTTTTGAAGATCCCGATATCTTCACAATGGATATCAGAGGAAATGTATTA |
| | AAAAATATCCGGATTTTTGCTCCTTCTTATGTGATCAAAAACAAACGCTTTGATATTACA |
| | GTGCGCTTCGAAGATGAATTTGGGAACTTAACCAATTTCTCCCCAGAAGAGACCCATATC |
| | GAGCTTTCGTACGAACATCTTCGCGAAAACCTCAATTGGCAATTGTTCATCCCTGAAACA |
| | GGCTTTGTGATCCTTCCAAACCTGTATTTCAATGAACCAGGTATTTATCGTATTCAACTA |
| | CGCAATCAAGCAACAAAAGAGGTCTTTACATCAGCGCCTATCAAATGTTTTGCAGAAACC |
| | TCCTCTCATCTTTTGTGGGGGCTTCTACATGGAGAATCTGAACGTGTCGACTCTGAAGGT |
| | AATATCGAGTCTTGCTTGCGTTATTTTCGTGATGACTGCGCGTTAAACTTTTTTGCAACA |
| | TCCTCTTTCGAAATTCAAGATGGCCTGACCCCAGAAACCATTAAAACCATTAACCAAACC |
| | GTTGCTGATTTTAATGAAGAAGATCGTTTCATTGCCTTATCCGGAGCACAGTACCTTTCT |
| | GAAGAGCCTGGCGAGGGAATTCGTGAAGTATTGCTGATGAAGGAACCCAAATCCCCAGGG |
| | AAACATAAAGAATGCAAACTATTTCCTTTATCTAAGCTATATAAGCAATCAACTAGTCAT |
| | GAGTTAATCTCAATCCCCAGCTTCACTGCTTCAAAGAAATTTGGATACAATTTTAATAAT |
| | TTCCATCCTGAATTTGAAAGAGTTGTTGAAATTTATAATGCCTGGGGATGCTCTGAAAGA |
| | ACTGAAGCTGAAGGAAACCCTTTCCCTATTAAAGGTTCTATCGACTCAGAAAATCCAGAG |
| | GGAACTGTTCTATCTGCTTTAAAGAGAAACCTGCGTTTTGGATTCGTAGCCGGTGGTCTT |
| | GATGATAGAAATCTATACAATCACTTTTTTGATTCCGATCAACAGCAATACTCCCCTGGA |
| | TTAACAGCTGTGATCTGCAATAAATATTCTCGGGATTCCTTACTCGAGGCATTATACCAA |
| | CGACAATGCTATGCTACAACCGGCCAAAGAATTATCGTGAATTTCCAGATTACATCTGCT |
| | CCTATGGGCTCCGAACTCTCCACAGCCATTAAACCAGGGCTCGTGATCAATAGACATATT |
| | TCGGGATATGTAGCAGGAACTGCCAAGATTGCGTCGATCGAAATCATCCGCAATGAGGAT |
| | ATTCTCCATACCTTCCACCCAGATGGAAATAACTTTGAGTATGAGTACGACGATCTCTCT |
| | CCTTTTGCACAAGTCACTCTAAAAGATCCTCAAAATGGAGCTCCTTTTGCTTTTTACTAC |
| | TTACGAGTCACTCAAGAGAATGGAGCTATGGCTTGGAGCTCTCCTATTTGGATAGATCTT |
| | AACTAA (SEQ ID NO: 6) |
| CT497 | MATQTKKPQPTQLLSLPNSKESEMIVLGCMLTSVNHLNLAANLLQEDDFYFLEHRIIFRV |
| replicative DNA | LQDAFKSDRPMDPHLTGEELKRRDQLNIIGGPSYLITLSEFAGTSAYIEEYAEIIRSKSI |
| helicase | LRKMIQAAKDIEKKAAEEPRDVTTALDDAQNLLFRISQTTNLAPHVLVADKLKGVASSKD |
| gi\|15605226\|ref | KSFLLALQERQEAFQASAHDSSSPMLSGFPTHFLDLDKMISGFSPSNLIILAARPAMGKT |
| \|NP_220012.1\| | ALALNIVENFCFDSRLPVGIFSLEMTVDQLIHRIICSRSEVEAKKISVGDISGRDFQRVV |
| | SVVREMEEHTLLIDDYPGLKITDLRARARRMKESYDIQFLVIDYLQLISSSGNLRNSDSR |
| | NQEISEISRMLKNLARELNIPILCLSQLSRKVEDRANHRPLMSDLRESGSIEQDADQIMF |
| | LLRREYYDPNDKPGTAELIVAKNRHGSIGSVQLVFEKDFARFRNYAGCEFPG (SEQ ID |
| | NO: 7) |
| CT497 | ATGGCGACCCAGACAAAAAAACCCCAACCCACTCAACTCCTCTCTCTTCCTAATTCTAAG |
| gi\|15604717: | GAATCTGAAATGATTGTACTGGGGTGTATGTTGACCAGTGTCAATCATTTGAACCTTGCT |
| 575229-576647 | GCCAACCTTCTTCAGGAAGATGATTTCTACTTCTTAGAGCATCGTATTATTTTTCGTGTG |
| | TTGCAGGACGCTTTCAAGTCTGACCGCCCCATGGATCCCCATCTTACGGGAGAAGAACTC |
| | AAACGCCGAGATCAACTCAATATCATTGGAGGCCCCTCTTATCTAATCACTCTCTCTGAA |
| | TTTGCAGGGACATCTGCCTATATTGAAGAATACGCAGAAATTATCCGCTCCAAATCTATT |
| | CTAAGAAAAATGATTCAAGCGGCCAAAGATATCGAAAAGAAAGCCGCTGAAGAGCCACGT |
| | GACGTCACTACCGCTTTAGATGATGCTCAAAACTTATTGTTTCGCATAAGCCAAACAACT |
| | AACTTAGCCCCCCATGTCCTTGTTGCAGACAAACTCAAAGGAGTTGCTTCTTCTAAAGAC |
| | AAATCCTTCTTACTCGCTTTACAAGAACGCCAAGAAGCTTTCCAAGCAAGTGCTCATGAC |
| | TCAAGCTCTCCTATGCTCTCAGGCTTCCCCACTCATTTCTTAGATCTAGATAAAATGATT |
| | AGTGGGTTCAGCCCCTCTAACTTGATCATTCTTGCTGCTCGTCCTGCTATGGGGAAAACT |
| | GCTTTAGCTCTTAACATTGTAGAGAATTTTTGTTTTGATAGCCGGCTTCCTGTAGGAATT |
| | TTTTCATTAGAGATGACTGTAGACCAGTTAATCCATCGCATCATCTGCTCGCGTTCCGAA |
| | GTAGAAGCTAAAAAAATTAGCGTGGGAGATATTTCTGGTAGAGATTTTCAACGCGTGGTC |
| | TCTGTGGTAAGAGAGATGGAAGAACATACTCTACTCATAGATGACTACCCGGGATTGAAA |
| | ATCACAGATCTTCGAGCACGCGCAAGAAGAATGAAAGAAAGCTACGATATCCAATTTCTA |
| | GTTATTGACTACCTACAATTAATCTCTAGCTCTGGGAACCTAAGAAATTCTGATTCGCGA |
| | AACCAAGAAATTTCTGAAATCTCCAGAATGCTTAAAAATTTGGCTCGAGAACTCAATATT |
| | CCTATTCTCTGCCTCTCTCAATTATCTAGAAAAGTAGAAGACAGAGCTAATCACAGACCT |
| | TTGATGAGCGATTTAAGAGAAAGTGGAAGCATTGAACAAGATGCTGACCAAATTATGTTT |
| | TTACTTCGCCGCGAATATTATGATCCTAATGATAAACCTGGAACAGCAGAGTTGATTGTG |
| | GCTAAAAACCGCCACGGCTCCATTGGATCTGTACAATTAGTTTTTGAAAAAGACTTCGCT |
| | CGATTCCGAAATTATGCTGGCTGTGAGTTCCCTGGATAA (SEQ ID NO: 8) |
| CT843 | MSLDKGTKEEITKKFQLHEKDTGSADVQIAILTEHITELKEHLKRSPKDQNSRLALLKLV |
| 30S ribosomal | GQRRKLLEYLNSTDTERYKNLIARLNLRK (SEQ ID NO: 9) |
| protein S15 | |
| gi\|15605578\|ref | |
| NP_220364.1\| | |
| CT843 | ATGTCTTTGGATAAGGGCACTAAAGAAGAAATTACTAAAAAATTTCAACTTCATGAAAAA |
| gi\|15604717: | GACACAGGTTCGGCAGATGTGCAGATTGCTATTCTGACTGAGCACATAACGGAACTCAAG |
| 995075-995344 | GAGCACCTTAAAAGATCTCCTAAAGATCAAAATTCTCGTCTAGCTTTGCTAAAATTAGTA |
| | GGGCAGAGAAGAAAGCTCCTAGAGTACTTAAATTCTACTGATACTGAAAGATATAAAAAT |
| | TTAATTGCTCGCCTCAATTTGAGAAAATAG (SEQ ID NO: 10) |

CT209 polypeptide antigens, CT253 polypeptide antigens, CT425 polypeptide antigens, CT497 polypeptide antigens, and CT843 polypeptide antigens can be provided in any combination with each other and/or with other chlamydia antigens. In some embodiments, a combination of chlamydia polypeptide antigens includes two of CT209, CT253, CT425, CT497, and CT843 polypeptide antigens. In some embodiments, a combination includes three of CT209, CT253, CT425, CT497, and CT843 polypeptide antigens. In some embodiments, a combination includes four of CT209, CT253, CT425, CT497, and CT843 polypeptide antigens. In some embodiments, a combination includes CT209, CT253, CT425, CT497, and CT843 polypeptide antigens.

Other antigens which can be provided in combination with one or more of CT209, CT253, CT425, CT497, and CT843 polypeptide antigens, include one or more of CT062, CT104, CT144, CT111, CT242, CT491, CT601, CT687, CT732, CT781, CT788, CT808, and CT823 polypeptide antigens. In some embodiments, a combination of antigens includes one, two, three, four, or five of a CT209, CT253, CT425, CT497, or CT843 polypeptide antigen, and one, two, three, four, or five of a CT062, CT104, CT144, CT111, CT242, CT491, CT601, CT687, CT732, CT781, CT788, CT808, or CT823 polypeptide antigen. These antigens and specific epitopes of these antigens are described in PCT/US07/004675 (published as WO 2007/098255), PCT/US2008/0092, and PCT/US2008/013298, the entire contents of which are hereby incorporated by reference. Additional chlamydia polypeptide antigens that can be provided in combination with a novel antigen described herein include a polymorphic membrane protein D (PmpD or CT812; see GenBank NP_{—}220332.1 GI:15605546), a major outer membrane protein (MOMP or ompA or CT681; see GenBank NP_{—}220200.1 GI:15605414), CT858 or cpaf (GenBank NP 220380 GI:15605594), CT713 or PorB (GenBank NP_{—}220232.1 GI:15605446), OMP85 (GenBank NP_{—}219746.1 GI:15604962), CT315 or RpoB (GenBank NP_{—}219820.1 GI:15605036), CT316, CT737, or CT674. Sequences of the above-mentioned polypeptides, and nucleic acids that encode them, are known. See, e.g., a C. trachomatis genome sequence in GenBank under Acc. No. NC_{—}000117, GI:15604717, annotated genes, and linked polypeptide sequences therein.

The present invention also provides compositions that include a chlamydia antigen described herein and an antigen from a different infectious agent. In some embodiments, a composition includes a chlamydia antigen and an antigen from a different infectious agent that causes a sexually transmitted disease. In some embodiments, compositions that include a chlamydia antigen (e.g., a CT209 polypeptide antigen, a CT253 polypeptide antigen, a CT425 polypeptide antigen, a CT497 polypeptide antigen, a CT843 polypeptide antigen, or a combination thereof) and a papillomavirus antigen (e.g., a human papillomavirus antigen) are provided. In some embodiments, compositions that include a chlamydia antigen (e.g., a CT209 polypeptide antigen, a CT253 polypeptide antigen, a CT425 polypeptide antigen, a CT497 polypeptide antigen, a CT843 polypeptide antigen, or a combination thereof) and a herpesvirus antigen (e.g., a human herpes simplex virus-2 antigen) are provided. In some embodiments, compositions that include a chlamydia antigen (e.g., a CT209 polypeptide antigen, a CT253 polypeptide antigen, a CT425 polypeptide antigen, a CT497 polypeptide antigen, a CT843 polypeptide antigen, or a combination thereof) and a gonorrhea antigen are provided. In some embodiments, compositions that include a chlamydia antigen (e.g., a CT209 polypeptide antigen, a CT253 polypeptide antigen, a CT425 polypeptide antigen, a CT497 polypeptide antigen, a CT843 polypeptide antigen, or a combination thereof) and an antigen from one or more of a papillomavirus, a herpesvirus (e.g., HSV-2), and N. gonorrhoeae, are provided.

### Adjuvants

A large variety of formulations of immunogenic compositions can be employed to induce immune responses. A common route of administration in humans is by intramuscular (i.m.) injection, but immunogenic compositions may also be applied orally, intranasally, subcutaneously, by inhalation, intravenously, or by other routes of administration. In most cases, chlamydia antigens are initially presented to naive lymphocytes in regional lymph nodes.

In some embodiments, a chlamydia antigen composition includes purified components (e.g., purified antigens). In some embodiments, chlamydia antigens are fused to other molecules, such as proteins that can confer adjuvant activity, or moieties that facilitate isolation and purification (e.g., an epitope tag).

In some embodiments, a chlamydia antigen composition includes an adjuvant. In some embodiments, the adjuvant includes mineral-containing adjuvant. Mineral-containing ajduvants can be formulated as gels, in crystalline form, in amorphous form, as particles, etc. Mineral-containing adjuvants include, for example, aluminum salts and/or calcium salts (e.g., aluminum hydroxide, aluminum phosphate, aluminum sulfate, calcium phosphate, etc.). In some embodiments, a chlamydia antigen composition includes aluminum hydroxide. Alhydrogel™ is an example of an aluminum hydroxide gel adjuvant.

In some embodiments, an adjuvant includes an immunomodulatory oligonucleotide. In some embodiments, an immunomodulatory oligonucleotide sequence includes CpG (unmethylated cytosine-guanosine) motifs. Oligonucleotides having CpG motifs can include nucleotide analogs and/or non-naturally occurring internucleoside linkages (e.g., phosphorothioate linkages). For examples of various oligonucleotides include CpG motifs, see Kandimalla, et al., Nuc. Acids Res. 31(9): 2393-2400, 2003; WO02/26757; WO99/62923; Krieg, Nat. Med. 9(7): 831-835, 2003; McCluskie, et al., FEMS Immunol. Med. Microbiol. 32:179-185, 2002; WO98/40100; U.S. Pat. No. 6,207,646; U.S. Pat. No. 6,239,116 and U.S. Pat. No. 6,429,199. Other immunomodulatory nucleotide sequences double stranded RNA sequences, palindromic sequences, and poly(dG) sequences.

In some embodiments, an adjuvant comprises IC31™ (Intercell AG). IC31™ is a synthetic adjuvant that includes an antimicrobial peptide, KLK, and an immunostimulatory oligonucleotide, ODN1a, and acts as a Toll-like Receptor 9 (TLR9) agonist.

In some embodiments, an adjuvant includes a toxin. In some embodiments, a toxin is a bacterial ADP-ribosylating toxin, e.g., cholera toxin, E. coli heat labile toxin, or pertussis toxin. In some embodiments, the bacterial toxin is a detoxified form of an ADP-ribosylating toxin (see, e.g., Beignon, et al., Inf. Immun. 70(6):3012-3019, 2002; Pizza, et al., Vaccine 19:2534-2541, 2001; Pizza, et al., Int. J. Med. Microbiol. 290(4-5):455-461, 2000; Scharton-Kersten et al., Inf. Immun. 68(9):5306-5313, 2000; Ryan et al., Inf. Immun. 67(12):6270-6280, 1999; Partidos et al., Immunol. Lett. 67(3):209-216, 1999; Peppoloni et al., Vaccines 2(2):285-293, 2003; and Pine et al., J. Control Release 85(1-3):263-270, 2002).

In some embodiments, an adjuvant includes an endotoxin such as monophosphoryl lipid A or 3-De-O-acylated monophosphoryl lipid A (see U.S. Pat. No. 4,987,237 and GB 2122204B).

In some embodiments, an adjuvant includes a muramyl dipeptide (e.g., N-acetyl-muramyl-L-threonyl-D-isoglutamine(thr-MDP), N-acetyl-normuramyl-1-alanyl-d-isoglutamine(nor-MDP), and N-acetylmuramyl-1-alanyl-d-isoglutaminyl-1-alanine-2-(1′-2′-dipalmitoyl-s-n-glycero-3-hydroxyphosphoryloxy)-ethylamine MTP-PE).

In some, an adjuvant includes an oil emulsion and/or emulsifier-based adjuvant. In some embodiments, an oil emulsion adjuvant includes a Freund's Adjuvant (e.g., Complete Freund's adjuvant (CFA), or incomplete Freund's adjuvant (IFA)). In some embodiments, an oil-emulsion adjuvant includes a squalene water emulsion, such as MF59 (Novartis; see, e.g., WO9014837), or a Synex adjuvant formulation (SAF)). In some embodiments, an oil emulsion includes a dispersing agent, e.g., a mono- or di-C₁₂-C₂₄-fatty acid ester of sorbitan or mannide, e.g., sorbitan mono-stearate, sorbitan mon-oleate, or mannide mono-oleate. Examples of oil emulsions that include squalene and dispersing agents includes Arlacel™, Montanide™ ISA-720, and Montanide™ ISA-703. Other oil emulsions are described, e.g., in WO 95/17210 and EP 0399842.

In some embodiments, an adjuvant includes a saponin. Saponins are steroid and/or triterpenoid glycosides derived from plants such as Quillaja saponaria, Saponaria officianalis, Smilax ornata, and Gypsophilla paniculata. Fractions of saponin-containing extracts that have been described and that can be used as adjuvants for chlamydia antigens include Quil™A, QS21, QS7, QS17, QS18, QH-A, QH-B, QH-C, and QuilA (see, e.g., U.S. Pat. No. 5,057,540). In some embodiments, QS21 is used as an adjuvant.

In some embodiments, an adjuvant includes an immune stimulating complex (ISCOM). ISCOMs are particles that typically include a glycoside (e.g., a saponin) and a lipid. In some embodiments, an ISCOM includes a saponin and a cholesterol. In some embodiments, an ISCOM includes a saponin, a cholesterol, and a phospholipid (e.g., phosphatidylcholine and/or phosphatidylethanolamine). In some embodiments, an ISCOM includes a nonionic block copolymer. ISCOMs can include additional adjuvants, e.g., additional adjuvant substances described herein (see, e.g., WO 05/002620). In some embodiments, an ISCOM includes a substance that targets is to a mucosal members (see, e.g., WO97/030728). Other ISCOM compositions and preparation of the compositions suitable for combination with chlamydia antigens provided herein are described, e.g., in U.S. Pat. Pub. No. 20060121065, WO 00/07621, WO 04/004762, WO 02/26255, and WO 06/078213. In some embodiments, an adjuvant comprises an AbISCO® adjuvant (e.g., Matrix-M™, Isconova). In some embodiments, an adjuvant comprises AbISCO®-100. In some embodiments, an adjuvant comprises AbISCO®-300.

In some embodiments, an adjuvant includes a nonionic block copolymer. Nonionic block copolymers typically include two chains of hydrophobic polyoxyethylenes of various lengths combined with a block of hydrophobic polyoxypropylene. In some embodiments, a nonionic block copolymer is formulated in an oil-in-water emulsion (e.g., with oil and squalene).

In some embodiments, an adjuvant includes virus like particles (VLPs). VLPs are non replicating, non infectious particles that typically include one or more viral proteins, optionally formulated with an additional component such as a phospholipid. In some embodiments, a VLP includes proteins from one or more of the following: an influenza virus (e.g., a hemaglutinin (HA) or neuraminidase (NA) polyptide), Hepatitis B virus (e.g., a core or capsid polypeptide), Hepatitis E virus, measles virus, Sindbis virus, Rotavirus, Foot-and-Mouth Disease virus, Retrovirus, Norwalk virus, human papilloma virus, HIV, RNA-phages, Qβ-phage (e.g., a coat protein), GA-phage, fr-phage, AP205 phage, a Ty (e.g., retrotransposon Ty protein p1). See, e.g., WO03/024480, WO03/024481, WO08/061,243, and WO07/098,186.

In some embodiments, an adjuvant includes replicons. Replicons resemble VLPs in that they are noninfectious particles including viral proteins, and further include a nucleic acid encoding a polypeptide (e.g., an antigen). In some embodiments, a replicon includes proteins from an alphavirus. Alphaviruses include, e.g., Eastern Equine Encephalitis Virus (EEE), Venezuelan Equine Encephalitis Virus (VEE), Everglades Virus, Mucambo Virus, Pixuna Virus, Western Equine Encephalitis Virus (WEE), Sindbis Virus, Semliki Forest Virus, Middleburg Virus, Chikungunya Virus, O'nyong-nyong Virus, Ross River Virus, Barmah Forest Virus, Getah Virus, Sagiyama Virus, Bebaru Virus, Mayaro Virus, Una Virus, Aura Virus, Whataroa Virus, Babanki Virus, Kyzylagach Virus, Highlands J Virus, Fort Morgan Virus, Ndumu Virus, and Buggy Creek Virus. In some embodiments, an adjuvant includes a replicon that includes a nucleic acid encoding one or more chlamydia antigens described herein. In some embodiments, an adjuvant includes a replicon that encodes a cytokine (e.g., interleukin-12 (IL-12), IL-23, or granulocyte-macrophage colony-stimulating factor (GM-CSF)). Production and uses of replicons are described, e.g., in WO08/058,035, WO08/085,557, and WO08/033,966). In some embodiments, a VLP or replicon adjuvant includes one or more chlamydia antigens (i.e., VLP or replicon particles include a chlamydia antigen as part of the particles). In some embodiments, a VLP or replicon adjuvant is co-adminstered with a chlamydia antigen polypeptide.

In some embodiments, an adjuvant includes liposomes, which are artificially-constructed spherical lipid vesicles (see, e.g., U.S. Pat. Nos. 4,053,585; 6,090,406; and 5,916,588). In certain embodiments, a lipid to be used in liposomes can be, but is not limited to, one or a plurality of the following: phosphatidylcholine, lipid A, cholesterol, dolichol, sphingosine, sphingomyelin, ceramide, glycosylceramide, cerebroside, sulfatide, phytosphingosine, phosphatidyl-ethanolamine, phosphatidylglycerol, phosphatidylinositol, phosphatidylserine, cardiolipin, phosphatidic acid, and lyso-phosphatides. In some embodiments, an adjuvant includes a liposome and a ligand for a Toll-like Receptor (TLR) (see, e.g., WO/2005/013891, WO/2005/079511, WO/2005/079506, and WO/2005/013891). In some embodiments, an adjuvant includes JVRS-100. JVRS-100 comprises cationic liposomes combined with non-coding oligonucleotides or plasmids.

In some embodiments, an adjuvant includes microparticles comprised of a polymer, e.g., a polymer of acrylic or methacrylic acid, polyphosphazenes, polycarbonates, polylactic acid, polyglycolic acid, copolymers of lactic acid or glycolic acid, polyhydroxybutyric acid, polyorthoesters, polyanhydrides, polysiloxanes, polycaprolactone, or a copolymer prepared from the monomers of these polymers. In some embodiments, an adjuvant includes microparticles comprised of a polymer selected from the group consisting of polyvinylpyrrolidone, polyvinylalcohol, polyhydroxyethylmethacrylate, polyacrylamide, polymethacrylamide, and polyethyleneglycol (see, e.g., U.S. Pat. No. 5,500,161).

In some embodiments, an adjuvant includes biodegradable microspheres (e.g., microspheres comprised of poly(D,L-lactic acid), poly(D,L-glycolic acid), poly(ε-caprolactone), polye (α-hydroxy acid), polyhydroxybutyric acid, a polyorthoester, a polyanhydride, etc.).

In some embodiments, an adjuvant includes a cytokine. In some embodiments, an adjuvant includes IL-12. In some embodiments, an adjuvant includes IL-23. In some embodiments, an adjuvant includes GM-CSF.

### Nucleic Acid Compositions and Antigen Expression

Various types of vectors are suitable for expression of chlamydia antigens in an expression system (e.g., in a host cell). In some embodiments, a composition includes a vector suitable for expression in vitro (whether in a cell or in a cell-free system), e.g., for producing a polypeptide composition. The term “vector” refers to a nucleic acid molecule capable of transporting another nucleic acid to which it has been linked and can include, for example, a plasmid, cosmid or viral vector. The vector can be capable of autonomous replication or it can integrate into a host DNA. Viral vectors include, e.g., replication defective retroviruses, adenoviruses and adeno-associated viruses. Other types of viral vectors are known in the art.

A vector can include a nucleic acid encoding a chlamydia antigen in a form suitable for expression of the nucleic acid in a host cell. A recombinant expression vector typically includes one or more regulatory sequences operatively linked to the nucleic acid sequence to be expressed. Regulatory sequences include promoters, enhancers and other expression control elements (e.g., polyadenylation signals). Regulatory sequences include those which direct constitutive expression of a nucleotide sequence, as well as tissue-specific regulatory and/or inducible sequences. A sequence encoding a chlamydia antigen can include a sequence encoding a signal peptide (e.g., a heterologous signal peptide) such that the antigen is secreted from a host cell. The design of the expression vector can depend on such factors as the choice of the host cell to be transformed, the level of expression of protein desired, and the like.

Recombinant expression vectors can be designed for expression and production of chlamydia antigens in prokaryotic or eukaryotic cells. For example, antigens can be expressed in E. coli, insect cells (e.g., using baculovirus expression vectors), yeast cells or mammalian cells. Suitable host cells are discussed further in Goeddel, Gene Expression Technology: Methods in Enzymology 185, Academic Press, San Diego, Calif., 1990. Alternatively, a recombinant expression vector can be transcribed and translated in vitro, for example using T7 promoter regulatory sequences and T7 polymerase.

Expression of polypeptides in prokaryotes is often carried out in E. coli with vectors containing constitutive or inducible promoters directing the expression of either fusion or non-fusion proteins. Fusion vectors add a number of amino acids to a protein encoded therein, e.g., to the amino terminus or carboxy terminus of the recombinant protein, e.g., to increase expression of recombinant protein; to increase the solubility of the recombinant protein; and/or to aid in the purification of the recombinant antigen by acting as a ligand in affinity purification. Often, a proteolytic cleavage site is introduced at the junction of the fusion moiety and the recombinant antigen to enable separation of the recombinant antigen from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K. S. Gene 67:31-40, 1988), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Chlamydia antigen expression vectors provided herein include yeast expression vectors, vectors for expression in insect cells (e.g., a baculovirus expression vector) and vectors suitable for expression in mammalian cells.

An expression vector for use in mammalian cells can include viral regulatory elements. For example, commonly used promoters are derived from polyoma, Adenovirus 2, cytomegalovirus and Simian Virus 40. A vector can include an inducible promoter, e.g., a promoter regulated by a steroid hormone, by a polypeptide hormone (e.g., by means of a signal transduction pathway), or by a heterologous polypeptide (e.g., the tetracycline-inducible systems, “Tet-On” and “Tet-Off”; see, e.g., Clontech Inc., CA, Gossen and Bujard, Proc. Natl. Acad. Sci. USA 89:5547, 1992, and Paillard, Human Gene Therapy 9:983, 1989).

A host cell can be any prokaryotic or eukaryotic cell. For example, a chlamydia antigen can be expressed in bacterial cells (such as E. coli), insect cells, yeast or mammalian cells (such as Chinese hamster ovary cells (CHO) or COS cells (African green monkey kidney cells CV-1 origin SV40 cells; Gluzman, Cell 23:175-182, 1981)). Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation.

A host cell can be used to produce (i.e., express) a chlamydia antigen. Accordingly, the invention further provides methods for producing a chlamydia antigen using host cells. In one embodiment, the method includes culturing a host cell (into which a recombinant expression vector encoding a chlamydia antigen has been introduced) in a suitable medium such that a chlamydia antigen is produced. In another embodiment, the method further includes isolating a chlamydia antigen from the medium or the host cell. Purified chlamydia antigens can be used for administration to mammals to induce an immune response, and/or to generate antibodies specific for the antigens.

The present invention also provides nucleic acid compositions that encode chlamydia antigens for administration to a subject in vivo, e.g., to elicit an immune response to the antigen. In some embodiments, a nucleic acid composition for administration in vivo includes a naked DNA plasmid encoding a chlamydia antigen. Bacterial vectors, replicon vectors, live attenuated bacteria, and viral vectors for expression of heterologous genes also can be used. Live attenuated viral vectors (e.g., recombinant vaccinia (e.g., modified vaccinia Ankara (MVA), IDT Germany), recombinant adenovirus, avian poxvirus (e.g., canarypox (e.g., ALVAC™, Aventis Pasteur) or fowlpox), poliovirus, and alphavirus virion vectors) have been successful in inducing cell-mediated immune response to antigens. Avian poxviruses are defective in mammalian hosts, but can express inserted heterologous genes under early promoters. Recombinant adenovirus and poliovirus vectors can thrive in the gut and so can stimulate efficient mucosal immune responses. Finally, attenuated bacteria can also be used as a vehicle for DNA vaccine delivery. Examples of suitable bacteria include S. enterica, S. tymphimurium, Listeria, and BCG. The use of mutant bacteria with weak cell walls can aid the exit of DNA plasmids from the bacterium.

Nucleic acid compositions used for immunization can include an adjuvant (e.g., an adjuvant such as a polymer, a saponin, muramyl dipeptide, liposomes, immunomodulatory oligonucleotide, or another adjuvant described herein) to promote nucleic acid uptake. Regardless of route, adjuvants can be administered before, during, or after administration of the nucleic acid. In some embodiments, an adjuvant increases the uptake of nucleic acid into host cells and/or increases expression of the antigen from the nucleic acid within the cell, induce antigen presenting cells to infiltrate the region of tissue where the antigen is being expressed, or increase the antigen-specific response provided by lymphocytes.

### Antibodies

This invention provides, inter alia, antibodies, or antigen-binding fragments thereof, to a novel chlamydia antigen described herein, e.g., a CT209 polypeptide antigen, a CT253 polypeptide antigen, a CT425 polypeptide antigen, a CT497 polypeptide antigen, or a CT843 polypeptide antigen. The antibodies can be of the various isotypes, including: IgG (e.g., IgG1, IgG2, IgG3, IgG4), IgM, IgA1, IgA2, IgD, or IgE. In some embodiments, an antibody is an IgG isotype, e.g., IgG1. An antibody against a chlamydia antigen can be full-length (e.g., an IgG1 or IgG4 antibody) or can include only an antigen-binding fragment (e.g., a Fab, F(ab)2, Fv or a single chain Fv fragment). These include monoclonal antibodies, recombinant antibodies, chimeric antibodies, human antibodies, and humanized antibodies, as well as antigen-binding fragments of the foregoing.

Monoclonal antibodies can be produced by a variety of techniques, including conventional monoclonal antibody methodology, e.g., the standard somatic cell hybridization technique of Kohler and Milstein, Nature 256: 495, 1975. Polyclonal antibodies can be produced by immunization of animal or human subjects. See generally, Harlow, E. and Lane, D. Antibodies: A Laboratory Manual, Cold Spring Harbor Laboratory Press, Cold Spring Harbor, N.Y., 1988. Antibodies against chlamydia antigens described herein can be used, e.g., for diagnostic assays, or for therapeutic applications.

In some embodiments of the present invention, a subject's response to an immunogenic composition described herein is evaluated, e.g., to determine efficacy of the composition, and/or to compare responses elicited by the composition to responses elicited by a different composition.

### Assays for T Cell Activation

In some embodiments, various assays can be utilized in order to characterize an antigen or composition and/or to determine whether an immune response has been stimulated in a T cell or group of T cells. In some embodiments, assays are used to characterize a T cell response in a subject that has been administered an immunogenic composition to elicit an anti-chlamydia response (e.g., to evaluate whether a detectable T cell response has been elicited and/or to evaluate the potency of the response). The novel chlamydia antigens described herein also provide diagnostic agents to evaluate exposure to chlamydia infections (e.g., in non-vaccinated subjects). In some embodiments, assays are used to characterize a T cell response in a subject to determine whether the subject has been infected with a chlamydia organism. The subject can be a subject suspected of exposure to a chlamydia organism recently (i.e., an assay to detect a response can be performed with a sample taken from the subject about 3, 4, 5, 6, 7, 8, 9, 10, 14, 30, or more days after suspected exposure to a chlamydia organism). The subject can be a subject suspected of exposure to a chlamydia organism weeks, months, or years prior to the assay.

In some embodiments, stimulation of an immune response in T cells is determined by measuring antigen-induced production of cytokines by T cells. In some embodiments, stimulation of an immune response in T cells can be determined by measuring antigen-induced production of IFNγ, IL-4, IL-2, IL-6, IL-10, IL-17 and/or TNFα by T cells. In some embodiments, antigen-induced production of cytokines by T cells can be measured by intracellular cytokine staining followed by flow cytometry. Other suitable methods include surface capture staining followed by flow cytometry, or methods that determine cytokine concentration in supernatants of activated T cell cultures, such as ELISA or ELISPOT assays.

In some embodiments, antigen-produced production of cytokines by T cells is measured by ELISPOT assay. ELISPOT assays typically employ a technique very similar to the sandwich enzyme-linked immunosorbent assay (ELISA) technique. An antibody (e.g. monoclonal antibody, polyclonal antibody, etc.) is coated aseptically onto a PVDF (polyvinylidene fluoride)-backed microplate. Antibodies are chosen for their specificity for the cytokine of interest. The plate is blocked (e.g., with a serum protein that is non-reactive with any of the antibodies in the assay). Cells to be tested for cytokine production are plated out at varying densities, along with antigen or mitogen, and then placed in a humidified 37° C. CO₂ incubator for a specified period of time. Cytokine secreted by activated cells is captured locally by the coated antibody on the high surface area PVDF membrane. After washing the wells to remove cells, debris, and media components, a secondary antibody (e.g. a biotinylated polyclonal antibody) specific for the cytokine is added to the wells. This antibody is reactive with a distinct epitope of the target cytokine and thus is employed to detect the captured cytokine. Following a wash to remove any unbound biotinylated antibody, the detected cytokine is then visualized using an avidin-HRP, and a precipitating substrate (e.g., AEC, BCIP/NBT). The colored end product (a spot, usually a blackish blue) typically represents an individual cytokine-producing cell. Spots can be counted manually (e.g., with a dissecting microscope) or using an automated reader to capture the microwell images and to analyze spot number and size. In some embodiments, each spot correlates to a single cytokine-producing cell.

In some embodiments, an immune response in T cells is said to be stimulated if between about 1% and about 100% of antigen-specific T cells produce cytokines. In some embodiments, an immune response in T cells is said to be stimulated if at least about 1%, at least about 5%, at least about 10%, at least about 25%, at least about 50%, at least about 75%, at least about 90%, at least about 95%, at least about 99%, or about 100% of antigen-specific T cells produce cytokines.

In some embodiments, an immune response in T cells is said to be stimulated if immunized subjects comprise at least about 10-fold, at least about 50-fold, at least about 100-fold, at least about 500-fold, at least about 1000-fold, at least about 5000-fold, at least about 10,000-fold, at least about 50,000-fold, at least about 100,000-fold, or greater than at least about 100,000-fold more cytokine-producing cells than do naïve controls.

In some embodiments, stimulation of an immune response in T cells can be determined by measuring antigen-induced proliferation of T cells. In some embodiments, antigen-induced proliferation may be measured as uptake of H³-thymidine in dividing T cells (sometimes referred to as “lymphocyte transformation test, or “LTT”). In some embodiments, antigen-induced proliferation is said to have occurred if ³H-thymidine uptake (given as number of counts from a γ counter) is at least about 5-fold, at least about 10-fold, at least about 20-fold, at least about 50-fold, at least about 100-fold, at least about 500-fold, at least about 1000-fold, at least about 5000-fold, at least about 10,000-fold, or greater than at least about 10,000-fold higher than a naïve control.

In some embodiments, antigen-induced proliferation may be measured by flow cytometry. In some embodiments, antigen-induced proliferation may be measured by a carboxyfluorescein succinimidyl ester (CFSE) dilution assay. CFSE is a non-toxic, fluorescent, membrane-permeating dye that binds the amino groups of cytoplasmic proteins with its succinimidyl-reactive group (e.g., T cell proteins). When cells divide, CFSE-labeled proteins are equally distributed between the daughter cells, thus halving cell fluorescence with each division. Consequently, antigen-specific T cells lose their fluorescence after culture in the presence of the respective antigen (CFSE^{low}) and are distinguishable from other cells in culture (CFSE^{high}). In some embodiments, antigen-induced proliferation is said to have occurred if CFSE dilution (given as the percentage of CFSE^{low} cells out of all CFSE⁺ cells) is at least about 5%, at least about 10%, at least about 25%, at least about 50%, at least about 75%, at least about 90%, at least about 95%, or at least about 100%.

In some embodiments, an immune response in T-cells is said to be stimulated if cellular markers of T cell activation are expressed at different levels (e.g., higher or lower levels) relative to unstimulated cells. In some embodiments, CD11a CD27, CD25, CD40L, CD44, CD45RO, and/or CD69 are more highly expressed in activated T cells than in unstimulated T cells. In some embodiments, L-selectin (CD62L), CD45RA, and/or CCR7 are less highly expressed in activated T cells than in unstimulated T cells.

In some embodiments, an immune response in T cells is measured by assaying cytotoxicity by effector CD8 T cells against antigen-pulsed target cells. For example, a ⁵¹chromium (⁵¹Cr) release assay can be performed. In this assay, effector CD8 T cells bind infected cells presenting virus peptide on class I MHC and signal the infected cells to undergo apoptosis. If the cells are labeled with ⁵¹Cr before the effector CD8 T cells are added, the amount of ⁵¹Cr released into the supernatant is proportional to the number of targets killed. In some embodiments, an immune response in T cells is measured by detecting expression of one or more of Perforin, Granzyme B, or CD107a (e.g., by ELISPOT or flow cytometry). See, e.g., Betts et al., J. Immunol. Meth. 281(1-2):65-78, 2003.

### Assays for B Cell Activation

In some embodiments, various assays can be utilized in order to determine whether an immune response has been stimulated in a B cell or group of B cells, e.g., to characterize an antibody response in a subject that has been administered an immunogenic composition against chlamydia, or to determine whether a subject has been exposed to a chlamydia organism. In some embodiments, stimulation of an immune response in B cells can be determined by measuring antibody titers. In general, “antibody titer” refers to the ability of antibodies to bind antigens at particular dilutions. For example, a high antibody titer refers to the ability of antibodies to bind antigens even at high dilutions. In some embodiments, an immune response in B cells is said to be stimulated if antibody titers are measured to be positive at dilutions at least about 5-fold greater, at least about 10-fold greater, at least about 20-fold greater, at least about 50-fold greater, at least about 100-fold greater, at least about 500-fold greater, at least about 1000 fold greater, or more than about 1000-fold greater than in non-immunized individuals or pre-immune serum.

In some embodiments, stimulation of an immune response in B cells can be determined by measuring antibody affinity. In particular, an immune response in B cells is said to be stimulated if an antibody that has an equilibrium dissociation constant (K_{d}) less than 10⁻⁷ M, less than 10⁻⁸ M, less than 10⁻⁹ M, less than 10⁻¹⁰ M, less than 10⁻¹¹ M, less than 10⁻¹² M, or less, has been elicited.

In some embodiments, a T cell-dependent immune response in B cells is said to be stimulated if class-switch recombination has occurred. In particular, a switch from IgM to another isotype (e.g., to an IgG isotype or to IgA or to a mixture of these isotypes) is indicative of a T-cell dependent immune response in B cells.

In some embodiments, an immune response in B cells is determined by measuring affinity maturation of antigen-specific antibodies. Affinity maturation occurs during the germinal center reaction whereby activated B cells repeatedly mutate a region of the immunoglobulin gene that encodes the antigen-binding region. B cells producing mutated antibodies which have a higher affinity for antigen are preferentially allowed to survive and proliferate. Thus, over time, the antibodies made by B cells in GCs acquire incrementally higher affinities. In some embodiments, the readout of this process is the presence of high antibody titer (e.g. high affinity IgG antibodies that bind and neutralize antigens even at high dilutions).

In some embodiments, an immune response in B cells is said to be stimulated if memory B cells and/or long-lived plasma cells that can produce large amounts of high-affinity antibodies for extended periods of time have formed. In some embodiments, antibody titers are measured after different time intervals (e.g. 2 weeks, 1 month, 2 months, 6 months, 1 year, 2 years, 5 years, 10 years, 15 years, 20 years, 25 years, or longer) after vaccination in order to test for the presence of memory B cells and/or long-lived plasma cells that can produce large amounts of high-affinity antibodies for extended periods of time. In some embodiments, memory B cells and/or long-lived plasma cells that can produce large amounts of high-affinity antibodies for extended periods of time are said to be present by measuring humoral responses (e.g. if humoral responses are markedly more rapid and result in higher titers after a later booster vaccination than during the initial sensitization).

In some embodiments, an immune response in B cells is said to be stimulated if a vigorous germinal center reaction occurs. In some embodiments, a vigorous germinal center reaction can be assessed visually by performing histology experiments. In some embodiments, vigorous germinal center reaction can be assayed by performing immunohistochemistry of antigen-containing lymphoid tissues (e.g., vaccine-draining lymph nodes, spleen, etc.). In some embodiments, immunohistochemistry is followed by flow cytometry.

In some embodiments, stimulation of an immune response in B cells can be determined by identifying antibody isotypes (e.g., IgG, IgA, IgE, IgM). In certain embodiments, production of IgG isotype antibodies by B cells is a desirable immune response by B cells. In certain embodiments, production of IgA isotype antibodies by B cells is a desirable immune response by B cells.

In some embodiments, an immune response in B cells is determined by analyzing antibody function in neutralization assays. In one example, the ability of a chlamydia organism to infect a susceptible cell in vitro in the absence of serum is compared to conditions when different dilutions of immune and non-immune serum are added to the culture medium in which the cells are grown. In certain embodiments, an immune response in a B cell is said to be stimulated if infection by a chlamydia organism is neutralized at a dilution of about 1:5, about 1:10, about 1:50, about 1:100, about 1:500, about 1:1000, about 1:5000, about 1:10,000, or less. Assays for neutralization of chlamydia are described, e.g., in Peeling et al., Infect. Immun. 46:484-488, 1984; and Peterson et al., Infect. Immun. 59:4147-4153, 1991.

### In Vivo Assays

In some embodiments, an immunogenic composition may be characterized (e.g., to assess efficacy in inducing a beneficial response in animal models) by infecting groups of immunized and non-immunized mice (e.g., 3 or more weeks after vaccination) with a dose of a chlamydia organism that typically produces a particular pathology (e.g., upper urogenital tract infection) or bacterial burden. The magnitude and duration of pathology or bacterial burden due to infection of both groups is monitored and compared. In one example, B cell responses are characterized by transferring serum from immune mice as a “passive vaccine” to assess protection of non-immune mice from pathological effects or burden of infection. In some embodiments, infiltrating leukocyte populations are characterized (e.g., to assess the number and type cells in a region of infection, e.g., whether CD4 T cells, CD8 T cells, or other cell types are present). Animal models for chlamydial urogenital infection have been described. In some embodiments, a chlamydia organism is applied as an intravaginal inoculum, and infection and pathology of one or more of lower and upper genital tracts of the infected animal is characterized. See, e.g., Barron et al. (J. Infect. Dis. 143(1):63-6, 1981), which describes an intravaginal infection model in mice. In some embodiments, clearance of primary infection is a measure of protective immunity in this model. In some embodiments, detection of CD4 T cell responses of a Th1 subtype correlate with protection (Morrison et al., Infect. Immun. 70:2741-2751, 2002).

In some embodiments, an immunogenic composition is assessed in an animal model of chlamydia infection. In some embodiments, lower urogenital tract infection by chlamydia is assessed in the model (e.g., lower tract bacterial burden and/or inflammation due to infection is assessed). In some embodiments, upper tract infection by chlamydia is assessed in the model (e.g., one or more of upper tract bacterial burden, inflammation, infertility, collagen deposition, scarring due to infection, are assessed). In some embodiments, an ability to prevent ascension of a chlamydia infection from the lower tract to the upper genital tract is assessed. In some embodiments, rate of bacterial clearance from the lower tract is assessed. In some embodiments, rate of bacterial clearance from the upper tract is assessed. In some embodiments, an immunogenic composition is assessed in an animal model in multiple strains of the animal of interest (e.g., multiple mouse strains). In some embodiments, presence and size of hydrosalpinx (fluid blockage of fallopian tubes) is assessed.

In some embodiments, desirable immunogenic compositions are characterized as having one or more of the above effects in vivo (e.g., in an animal model). For example, in some embodiments, an immunogenic composition reduces lower urogenital tract infection by chlamydia bacteria. In some embodiments, an immunogenic composition reduces lower tract bacterial burden. In some embodiments, an immunogenic composition reduces lower tract inflammation due to infection. In some embodiments, an immunogenic composition reduces upper tract infection by chlamydia. In some embodiments, an immunogenic composition reduces one or more of upper tract bacterial burden, inflammation, infertility, collagen deposition, scarring due to a chlamydia infection. In some embodiments, an immunogenic composition reduces ascension of a chlamydia infection from the lower tract to the upper genital tract. In some embodiments, an immunogenic composition increases the rate of bacterial clearance from the lower tract and/or the upper tract. In some embodiments, an immunogenic composition reduces presence and/or size of hydrosalpinx or salpyngitis due to infection. In some embodiments, an immunogenic composition has one or more of the above effects in multiple animal strains (e.g., multiple mouse strains).

One of ordinary skill in the art will recognize that the assays described above are only exemplary methods which could be utilized in order to determine whether T cell activation and/or B cell activation has occurred. Any assay known to one of skill in the art which can be used to determine whether T and/or B cell activation has occurred falls within the scope of this invention. The assays described herein as well as additional assays that could be used to determine whether T and/or B cell activation has occurred are described in Current Protocols in Immunology (John Wiley & Sons, Hoboken, N.Y., 2007; incorporated herein by reference).

### Applications

The compositions and methods described herein can be used for the prophylaxis and/or treatment of any chlamydia infection, chlamydial disease, disorder, and/or condition. As used herein, “prophylaxis” refers to uses before onset of symptoms due to a chlamydia infection, chlamydial disease, disorder, and/or condition and/or before known exposure to a chlamydia organism. Subjects include, but are not limited to, humans and/or other primates; and other animals susceptible to infection by chlamydia organisms, including commercially relevant mammals such as cattle, pigs, horses, sheep, cats, and/or dogs; and/or birds, including commercially relevant birds such as chickens, ducks, geese, and/or turkeys.

In some embodiments, immunogenic compositions in accordance with the present invention may be used to treat, alleviate, ameliorate, relieve, delay onset of, inhibit progression of, reduce risk of infection by, and reduce severity of, and/or reduce incidence of one or more symptoms or features of a chlamydial disease, disorder, and/or condition. In some embodiments, inventive an immunogenic composition may be used to treat, alleviate, ameliorate, relieve, delay onset of, inhibit progression of, reduce severity of, and/or reduce incidence of one or more symptoms or features of chlamydial infection (e.g., C. trachomatis infection, C. pneumoniae infection, C. psittaci infection).

In one aspect of the invention, a method for the prophylaxis and/or treatment of chlamydia infection is provided. In some embodiments, the prophylaxis and/or treatment of chlamydia infection comprises administering a therapeutically effective amount of an immunogenic composition described herein to a subject in need thereof, in such amounts and for such time as is necessary to achieve the desired result. In certain embodiments of the present invention a “therapeutically effective amount” of an inventive immunogenic composition is that amount effective for reducing risk of infection by, or treating, alleviating, ameliorating, relieving, delaying onset of, inhibiting progression of, reducing severity of, and/or reducing incidence of one or more symptoms or features of chlamydia infection. A therapeutically effective amount may be determined on a population basis, and is not required to be an amount that naturally induces a protective response in a particular subject.

In some embodiments, inventive prophylactic and/or therapeutic protocols involve administering a therapeutically effective amount of one or more inventive immunogenic compositions to a healthy subject (i.e., a subject who does not display any symptoms of chlamydia infection and/or who has not been diagnosed with chlamydia infection). For example, healthy individuals may be vaccinated using inventive immunogenic compositions prior to development of chlamydia infection and/or onset of symptoms of chlamydia infection; at risk individuals (e.g., patients exposed to individuals suffering from chlamydia infection, patients at high risk for sexually transmitted diseases, individuals at risk due to young age (e.g., children, adolescents, or young adults)) can be treated substantially contemporaneously with (e.g., within 48 hours, within 24 hours, or within 12 hours of) the onset of symptoms of and/or exposure to chlamydia infection. Of course individuals known to have chlamydia infection may receive treatment at any time.

In some embodiments, inventive prophylactic and/or therapeutic protocols involve administering a therapeutically effective amount of one or more inventive immunogenic compositions to a subject such that an immune response is stimulated in both T cells and B cells.

In some embodiments, by combining one or more chlamydia antigens and adjuvants, immune responses (e.g. T cell responses) can be tailored to preferentially elicit the most desirable type of immune response for a given indication, e.g., humoral response, Th1 T cell response, cytotoxic T cell, response, and/or a combination of these responses.

### Immunogenic Compositions

The present invention provides immunogenic compositions (e.g., vaccines) comprising a novel chlamydia antigen, e.g., one or more of a CT209 polypeptide antigen, a CT253 polypeptide antigen, a CT425 polypeptide antigen, a CT497 polypeptide antigen, a CT843 polypeptide antigen, and one or more pharmaceutically acceptable excipients. In accordance with some embodiments, a method of administering an inventive immunogenic composition to a subject in need thereof is provided. In some embodiments, inventive compositions are administered to humans. For the purposes of the present invention, the phrase “active ingredient” generally refers to an inventive immunogenic composition comprising at least one chlamydia antigen and optionally comprising one or more additional agents, such as an adjuvant.

Although the descriptions of immunogenic compositions provided herein are principally directed to compositions which are suitable for administration to humans, it will be understood by the skilled artisan that such compositions are generally suitable for administration to animals of all sorts. Modification of immunogenic compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the immunogenic compositions of the invention is contemplated include, but are not limited to, humans and/or other primates; mammals, including commercially relevant mammals such as cattle, pigs, horses, sheep, cats, and/or dogs; and/or birds, including commercially relevant birds such as chickens, ducks, geese, and/or turkeys.

The formulations of the immunogenic compositions described herein may be prepared by any method known or hereafter developed in the art of vaccines. In some embodiments, such preparatory methods include the step of bringing the antigen(s) (or nucleic acids encoding the antigens, for nucleic acid based applications) into association with one or more excipients and/or one or more other accessory ingredients, and then, if necessary and/or desirable, shaping and/or packaging the product into a desired single- or multi-dose unit.

An immunogenic composition of the invention may be prepared, packaged, and/or sold in bulk, as a single unit dose, and/or as a plurality of single unit doses. As used herein, a “unit dose” is discrete amount of the immunogenic composition comprising a predetermined amount of the antigen(s).

The relative amounts of the antigen(s), the pharmaceutically acceptable excipient(s), and/or any additional ingredients (e.g., adjuvant) in a composition of the invention will vary, depending upon the identity, size, and/or condition of the subject treated and further depending upon the route by which the composition is to be administered.

Immunogenic formulations of the present invention may additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired. Remington's The Science and Practice of Pharmacy, 21^{st} Edition, A. R. Gennaro, (Lippincott, Williams & Wilkins, Baltimore, Md., 2006; incorporated herein by reference) discloses various excipients used in formulating pharmaceutical compositions and known techniques for the preparation thereof. Except insofar as any conventional excipient is incompatible with a substance or its derivatives, such as by producing any undesirable biological effect or otherwise interacting in a deleterious manner with any other component(s) of the immunogenic composition, its use is contemplated to be within the scope of this invention.

In some embodiments, the pharmaceutically acceptable excipient is at least 95%, 96%, 97%, 98%, 99%, or 100% pure. In some embodiments, the excipient is approved for use in humans and for veterinary use. In some embodiments, the excipient is approved by United States Food and Drug Administration. In some embodiments, the excipient is pharmaceutical grade. In some embodiments, the excipient meets the standards of the United States Pharmacopoeia (USP), the European Pharmacopoeia (EP), the British Pharmacopoeia, and/or the International Pharmacopoeia.

Pharmaceutically acceptable excipients used in the manufacture of immunogenic compositions include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in the inventive formulations.

Injectable formulations, for example, sterile injectable aqueous or oleaginous suspensions may be formulated according to the known art using suitable dispersing or wetting agents and suspending agents. A sterile injectable preparation may be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong release of an immunogenic composition and stimulate maximal uptake by antigen presenting cells in the vicinity of an injection site, it is often desirable to slow the absorption from subcutaneous or intramuscular injection. This may be accomplished by the use of a liquid suspension of crystalline or amorphous material with poor water solubility. Alternatively, delayed absorption of a parenterally administered drug form may be accomplished by dissolving or suspending the drug in an oil vehicle.

In some embodiments, an immunogenic composition is administered to a mucosal surface. Compositions for rectal or vaginal administration can include suppositories which can be prepared by mixing immunogenic compositions of this invention with suitable excipients such as cocoa butter, polyethylene glycol or a suppository wax which are solid at ambient temperature but liquid at body temperature and therefore melt in the rectum or vaginal cavity and release antigen.

In some embodiments, an immunogenic composition is administered orally. Solid dosage forms for oral administration include capsules, tablets, pills, powders, and granules. In such solid dosage forms, the antigen can be mixed with at least one inert, pharmaceutically acceptable excipient such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may comprise buffering agents.

Suitable devices for use in delivering immunogenic compositions by an intradermal route described herein include short needle devices such as those described in U.S. Pat. Nos. 4,886,499; 5,190,521; 5,328,483; 5,527,288; 4,270,537; 5,015,235; 5,141,496; and 5,417,662. Jet injection devices which deliver liquid immunogenic compositions to the dermis via a liquid jet injector and/or via a needle which pierces the stratum corneum and produces a jet which reaches the dermis are suitable. Jet injection devices are described, for example, in U.S. Pat. Nos. 5,480,381; 5,599,302; 5,334,144; 5,993,412; 5,649,912; 5,569,189; 5,704,911; 5,383,851; 5,893,397; 5,466,220; 5,339,163; 5,312,335; 5,503,627; 5,064,413; 5,520,639; 4,596,556; 4,790,824; 4,941,880; 4,940,460; and PCT publications WO 97/37705 and WO 97/13537. Ballistic powder/particle delivery devices which use compressed gas to accelerate an immunogenic composition in powder form through the outer layers of the skin to the dermis are suitable. Alternatively or additionally, conventional syringes may be used in the classical mantoux method of intradermal administration.

General considerations in the formulation and/or manufacture of pharmaceutical agents may be found, for example, in Remington: The Science and Practice of Pharmacy 21^{st} ed., Lippincott Williams & Wilkins, 2005.

### Administration

In some embodiments, a therapeutically effective amount of an inventive immunogenic composition is delivered to a patient and/or animal prior to, simultaneously with, and/or after exposure to a chlamydia organism or diagnosis with a chlamydial disease, disorder, and/or condition. In some embodiments, a therapeutic amount of an inventive composition is delivered to a patient and/or animal prior to, simultaneously with, and/or after onset of symptoms of a chlamydial disease, disorder, and/or condition. In some embodiments, the amount of an immunogenic composition is sufficient to reduce risk of infection by, or treat, alleviate, ameliorate, relieve, delay onset of, inhibit progression of, reduce severity of, and/or reduce incidence of one or more symptoms or features of the chlamydial disease, disorder, and/or condition.

Immunogenic compositions, according to the method of the present invention, may be administered using any amount and any route of administration effective for treatment. The exact amount required will vary from subject to subject, depending on the species, age, and general condition of the subject, the severity of the infection, the particular composition, its mode of administration, its mode of activity, and the like. The specific effective dose level for any particular subject or organism will depend upon a variety of factors including the immunogenicity of the antigen composition employed; the specific composition employed; the nature of adjuvant used; the age, body weight, general health, sex and diet of the subject; the time of administration, route of administration, and like factors well known in the medical arts.

Immunogenic compositions of the present invention may be administered by any route that elicits an immune response. In some embodiments, an immunogenic composition is administered subcutaneously. In some embodiments, an immunogenic composition is administered intramuscularly. In some embodiments, the immunogenic compositions of the present invention are administered by a variety of routes, including oral, intravenous, intra-arterial, intramedullary, intrathecal, intraventricular, transdermal, interdermal, rectal, intravaginal, intraperitoneal, topical (as by powders, ointments, creams, and/or drops), transdermal, mucosal, nasal, buccal, enteral, sublingual; by intratracheal instillation, bronchial instillation, and/or inhalation; and/or as an oral spray, nasal spray, and/or aerosol.

In certain embodiments, an immunogenic composition of the invention may be administered in amounts that include a protein antigen in ranges of 1 μg-500 μg. In some embodiments, a dose of about 10 μg, 20 μg, 50 μg, or 100 μg is administered to a human.

In some embodiments, an immunogenic composition is administered more than once (e.g., twice, three times, four times, five times). In some embodiments, a boost is given about 2 weeks, one month, three months, six months, one year, or longer after an initial immunization.

### Kits

The present invention provides a variety of kits comprising one or more of the antigens described herein. For example, the invention provides a kit including a novel chlamydia antigen and instructions for use. A kit may include multiple different chlamydia antigens. A kit may include any of a number of additional components or reagents in any combination. All of the various combinations are not set forth explicitly but each combination is included in the scope of the invention.

According to certain embodiments of the invention, a kit may include, for example, (i) an immunogenic composition including at least one of the following chlamydia antigens: CT209, CT253, CT425, CT497, or CT843 polypeptide antigens; and (ii) instructions for administering the composition to a subject in need thereof. In some embodiments, the kit further includes an adjuvant.

Kits that include nucleic acids encoding chlamydia antigens are also provided. In certain embodiments, a kit may include, for example, (i) a composition including a nucleic acid encoding a chlamydia antigen; (ii) instructions for use of the nucleic acid composition (e.g., instructions for expressing the nucleic acid for producing the antigen, or instructions for administering the composition to a subject in need thereof to elicit a response against chlamydia).

Instructions included with kits may, for example, include protocols and/or describe conditions for production of immunogenic compositions and/or administration of immunogenic compositions, to a subject in need thereof, etc. Kits generally include one or more vessels or containers so that some or all of the individual components and reagents may be separately housed. Kits may also include a means for enclosing individual containers in relatively close confinement for commercial sale, e.g., a plastic box, in which instructions, packaging materials such as styrofoam, etc., may be enclosed. An identifier, e.g., a bar code, radio frequency identification (ID) tag, etc., may be present in or on the kit or in or one or more of the vessels or containers included in the kit. An identifier can be used, e.g., to uniquely identify the kit for purposes of quality control, inventory control, tracking, movement between workstations, etc.

### EXEMPLIFICATION

### Example 1

### Identification of Chlamydia trachomatis Antigens

### Isolation of Chlamydia-Specific T Cells

Chlamydia specific T cells were elicited by the intraperitoneal (i.p.) administration of 10⁷ infectious units of Chlamydia trachomatis elemental bodies (EB) into inbred mice. Six days post injection, the mice were euthanized and the peritoneal elicited cells (PEC) harvested by lavaging the cavity with PBS containing penicillin/streptomycin. The PECs were pooled from each mouse and the T cell populations enriched by magnetic bead depletion using the Miltenyi Pan T sorting kit following the manufacturer's instructions. The resulting enriched T cell population was then sorted using antibody-conjugated magnetic beads specific for CD4⁺ T cells (Miltenyi). The CD4 negative population was considered CD8⁺. Both T cell subsets were expanded non-specifically in vitro using either plate bound anti-CD3 and anti-CD28 antibodies (5 μg/mL each) or Dynabeads' Mouse CD3/CD28 T cell expander kit. The T cells were maintained at 10⁶ cells/mL in cPRMI-10% (RPMI, 10% FCS, HEPES, NEAA, Sodium Pyruvate, L-glutamine, penicillin/streptomycin and beta-mercaptoethanol) plus IL-2.

In Vitro Chlamydia trachomatis Library Screen with Expanded Cells

Six days prior to the library screen, naïve syngeneic mice were injected i.p. with 1 mL of 3% brewer thioglycollate yeast to elicit macrophages. The day before the screen, the mice were euthanized and their PECs harvested and pooled as described above. The recovered PECs (predominantly macrophages) were resuspended in cRPMI-10%, and then plated at 10⁵ cells per well in flat-bottom 96 well plates and placed into a 37° C., 5% CO₂ humidified incubator overnight.

Expanded cells were screened using a chlamydia ORFeome library. The ORFeome library is a set of E. coli that express each open reading frame in the C. trachomatis serovar D/UW-3/Cx genome, which was prepared as described in WO 2007/098255, which is herein incorporated by reference. The next day, the chlamydia ORFeome library was removed from the freezer, allowed to thaw at room temperature, and added to the macrophages at a 100:1 ratio of induced bacteria to macrophage, and returned to the 37° C., 5% CO₂ humidified incubator. After a two hour incubation, the cells were washed with PBS and then fixed with 1% paraformaldehyde (PFA). The cells were washed extensively, and residual PFA was quenched with 120 mM Lysine. After a final rinse with PBS, the expanded T cells were added to the pulsed, fixed macrophages at 10⁵ cells per well. The plates containing the expanded T cells and pulsed macrophages were returned to the 37° C., 5% CO₂ humidified incubator. Twenty-four hours later, cell-free supernatants were harvested and evaluated for the presence of IFNγ via ELISA.

Identification of Novel Chlamydia trachomatis Antigens in Mice

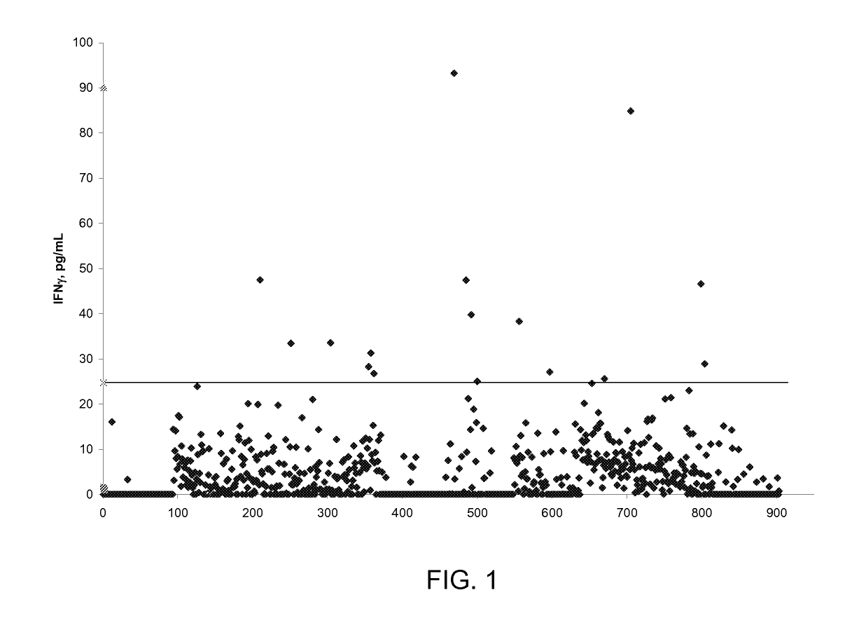

The library screen was performed using cells from several inbred mouse strains including Balb/c, C57BL/6, C3H and one out bred strain, CD1. Based on the results of each ELISA, clones that stimulated T cells to produce an IFNγ response greater than two standard deviations above the mean of all data were considered to be significant; sample data from CD8 T cells from C57BL/6 mice are shown in FIG. 1. The T cell response to each clone in the library is represented by a diamond, and the cutoff of the mean of all the data plus 2×SD is indicated by the line. For this screen (FIG. 1), approximately seventeen antigens out of the 894 possible proteins elicited responses above the cutoff.

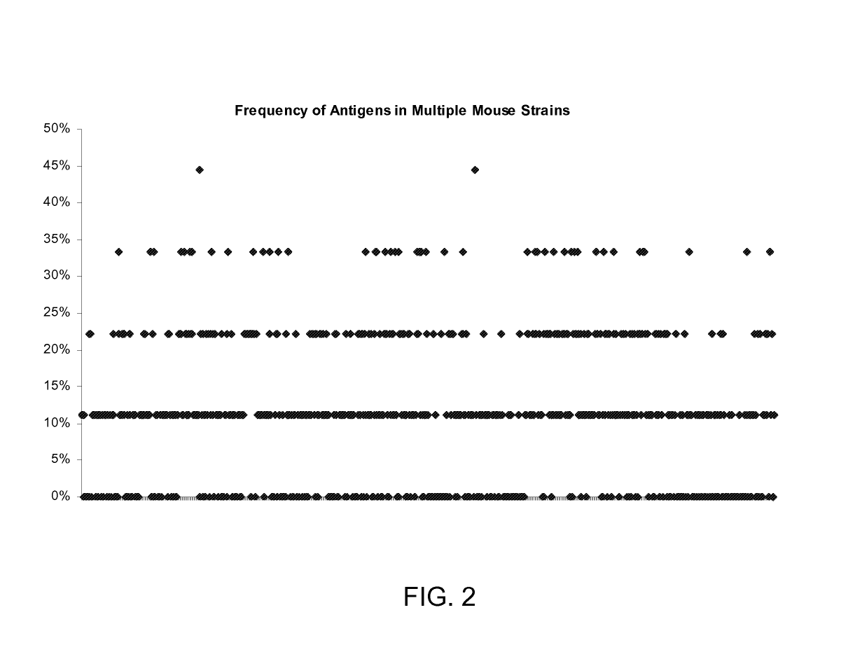

The complete data set of each library screen from multiple strains of mice was subsequently compared to determine the frequency at which Chlamydia trachomatis antigens significantly induced a T cell response (FIG. 2). Further subdividing the data based upon the induction of CD4 or CD8 T cells (or both), and comparing statistical and total frequencies across all strains of mice, five novel Chlamydia proteins were identified as immunogenic antigens: CT209, CT253, CT425, CT843 and CT497. The most frequently identified antigens across multiple strains of mice that had been previously infected with C. trachomatis serovar D, their function, and location in the bacterium are listed in Table 2.

TABLE 2
Five Chlamydia antigens identified in library screens.

| Clone location | | |
|---|---|---|
| in bacteria | Predicted Function | Predicted |
| CT209 | leucyl-tRNA synthetase | cytoplasm |
| CT253 | hypothetical protein | unknown |
| CT425 | hypothetical protein | unknown |
| CT497 | replicative DNA helicase | nucleus |
| CT843 | 30S ribosomal protein S15 | cytoplasm |

To validate the identity of the antigens, plasmid DNA from the library stocks was purified and sequenced. The resulting sequences are listed in Table 3, which shows the sequencing data for the genes that encoded T cell antigens identified in the library. The primer used for sequencing was a consensus primer located within the plasmid, upstream of each clone. Query represents the actual sequence data from the plasmid insert; Sbjct is the sequence of the annotated gene found in GenBank. Alignments were performed using the nucleotide BLAST feature of the NCBI website on the internet at the following address: blast.ncbi.nlm.nih.gov/Blast.cgi.

TABLE 3
Sequence alignment of T cell antigen clones.

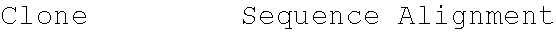

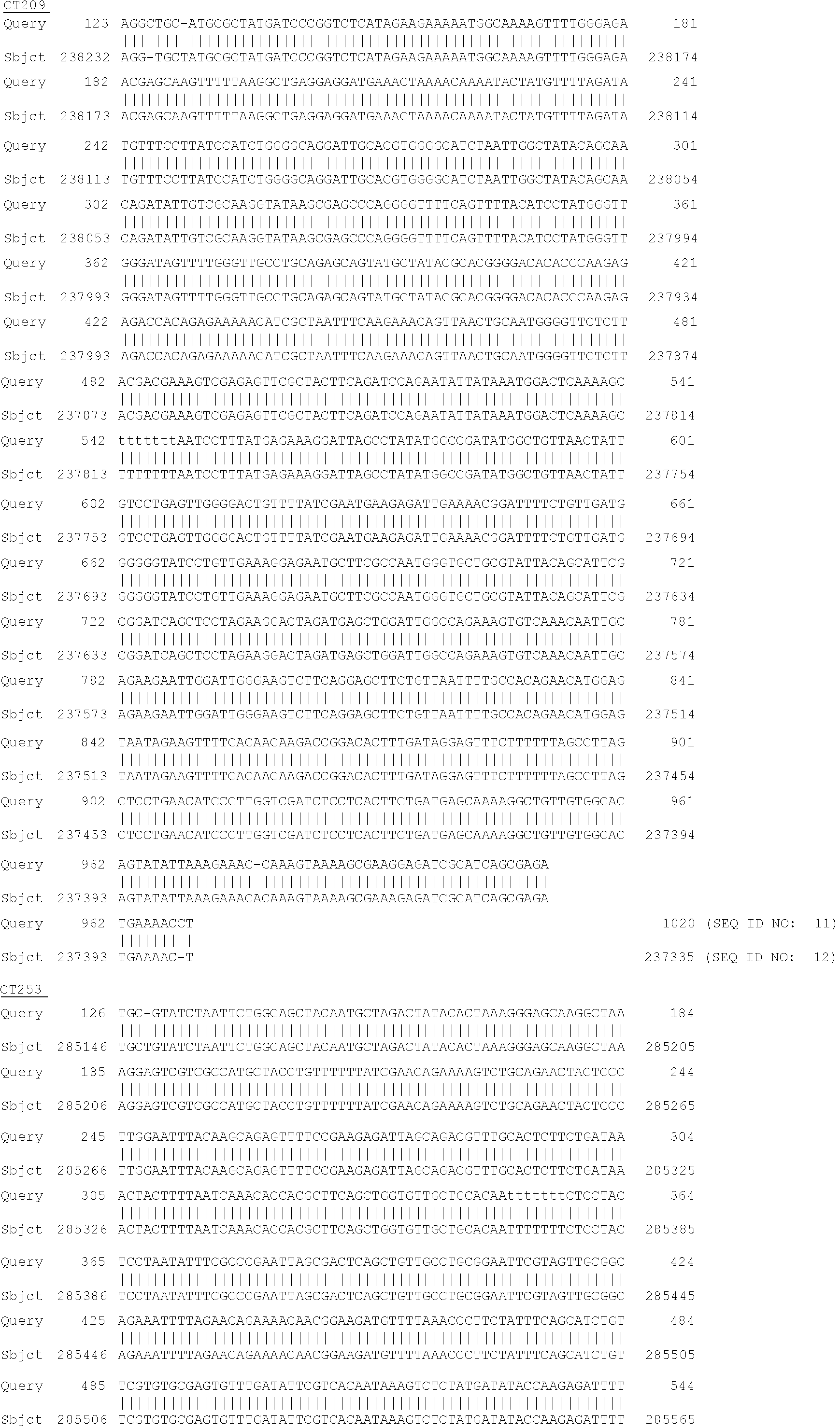

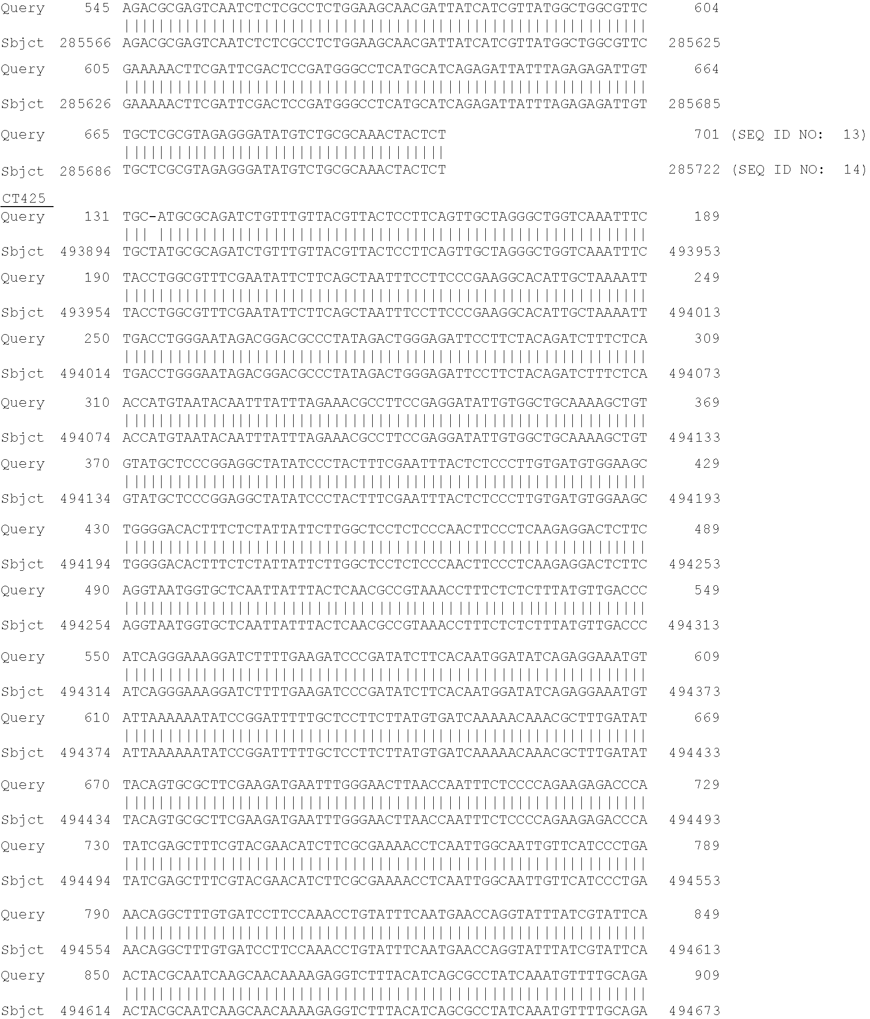

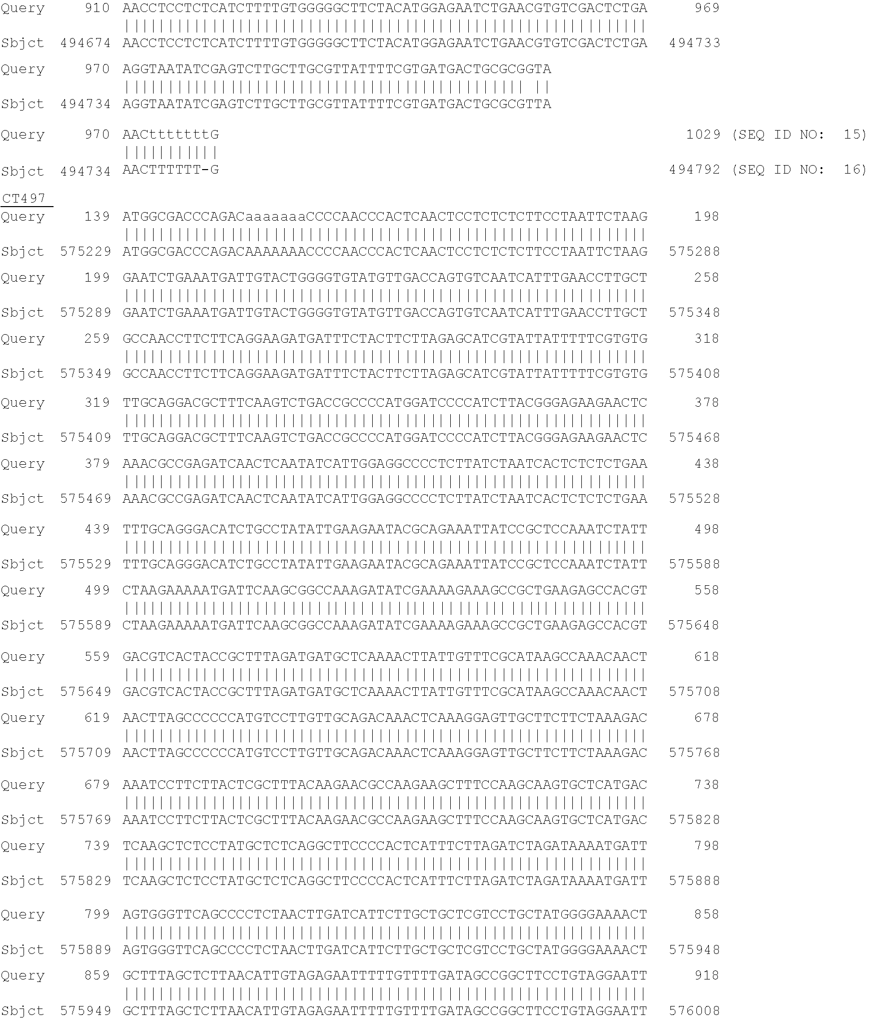

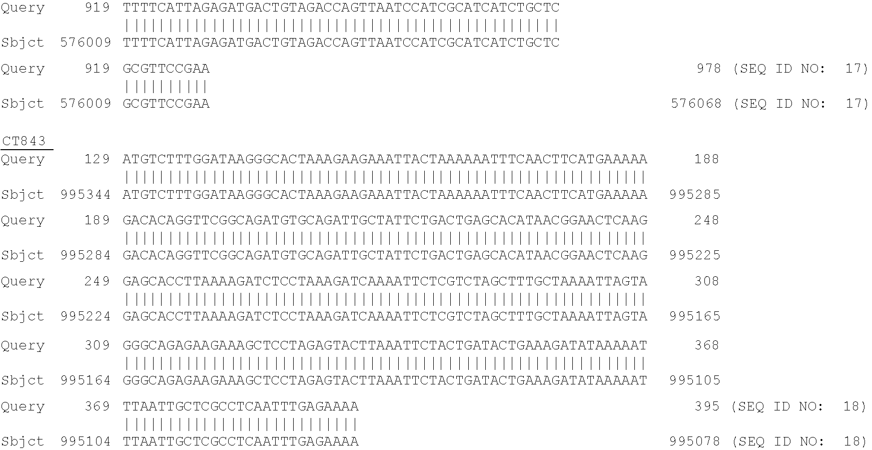

### Example 2

### Peripheral Blood Mononuclear Cells from Women with a Clinical History of Chlamydia Trachomatis Infection Respond to Identified Protein Antigens

Isolation and Screening of Chlamydia-Specific T Cells from Humans

Heparinized whole blood or leukopack samples were collected from humans with a documented clinical history of genital Chlamydia trachomatis infection or who have had multiple sexual exposures to an infected partner, and enriched by ficoll gradient centrifugation. CD4⁺ and CD8⁺ T cells and CD14⁺ monocytes were separated using antibody coated magnetic beads and placed into culture—the monocytes were derived into dendritic cells (MDDC) with GM-CSF and IL-4 cytokines, and the T cells were non-specifically expanded using magnetic beads coated with anti-CD3 and anti-CD28 antibodies plus recombinant IL-2. After sufficient cell numbers were achieved, the enriched and expanded CD4⁺ and CD8⁺ T cells were separately used to interrogate autologous MDDC that had been pulsed with an ORFeome library, to determine which antigens induced T cell responses naturally. The ORFeome library was the same library described in Example 1, except that it was moved to an E. coli strain that was inducible with arabinose rather than bacteriophage. After 18 hours of coculture, T cell responses were monitored by measuring interferon gamma (IFNγ) in the cell-free supernatants by ELISA.

Over 110 samples from human subjects were screened against the library. Library proteins that induced IFNγ responses that exceeded twice the mean average deviation of the median of the data after background correction were considered to be positive in this screen. Table 4 shows the frequencies with which the antigens were recognized by human donors. Frequencies were calculated by dividing the number of times a protein was positive by the number of subjects screened.

TABLE 4
Frequency of recognition of C. trachomatis
antigens by human donor CD4 or CD8 T cells

| Antigen | CD4 T cells | CD8 T cells |
|---|---|---|
| CT209 | 4% | 5% |
| CT253 | 6% | 2% |
| CT425 | 2% | 0% |
| CT497 | 9% | 12% |
| CT843 | 6% | 15% |

### EQUIVALENTS AND SCOPE

Those skilled in the art will recognize, or be able to ascertain using no more than routine experimentation, many equivalents to the specific embodiments of the invention, described herein. The scope of the present invention is not intended to be limited to the above Description, but rather is as set forth in the appended claims.

Those skilled in the art will recognize, or be able to ascertain using no more than routine experimentation, many equivalents to the specific embodiments of the invention described herein. The scope of the present invention is not intended to be limited to the above Description, but rather is as set forth in the appended claims.

In the claims articles such as “a,” “an,” and “the” may mean one or more than one unless indicated to the contrary or otherwise evident from the context. Thus, for example, reference to “a cell” includes reference to one or more cells known to those skilled in the art, and so forth. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where elements are presented as lists, e.g., in Markush group format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not been specifically set forth in haec verba herein. It is noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any antigen, any method of administration, any prophylactic and/or therapeutic application, etc.) can be excluded from any one or more claims, for any reason, whether or not related to the existence of prior art.

The publications discussed above and throughout the text are provided solely for their disclosure prior to the filing date of the present application. Nothing herein is to be construed as an admission that the inventors are not entitled to antedate such disclosure by virtue of prior disclosure.

1. An immunogenic composition comprising an isolated chlamydia antigen selected from a CT209 polypeptide antigen, a CT253 polypeptide antigen, a CT425 polypeptide antigen, a CT497 polypeptide antigen, a CT843 polypeptide antigen, and combinations thereof.

2-15. (canceled)

16. The composition of claim 1, wherein the composition comprises two or more chlamydia antigens.

17-22. (canceled)

23. The composition of claim 1, wherein the composition elicits an immune response to Chlamydia trachomatis.

24. The composition of claim 1, wherein the composition elicits a T cell mediated immune response to the chlamydia antigen.

25. The composition of claim 24, wherein the composition elicits a CD4 T cell mediated immune response to the chlamydia antigen.

26. The composition of claim 24, wherein the composition elicits a CD8 T cell mediated immune response to the chlamydia antigen.

27. The composition of claim 1, wherein the composition elicits an antibody response to the chlamydia antigen.

28. A method for eliciting an immune response against chlamydia in a mammal, the method comprising administering to the mammal an immunogenic composition comprising an isolated chlamydia antigen selected from a CT209 polypeptide antigen, a CT253 polypeptide antigen, a CT425 polypeptide antigen, a CT497 polypeptide antigen, a CT843 polypeptide antigen, and combinations thereof.

29. The method of claim 28, wherein the method elicits an immune response against Chlamydia trachomatis.

30. The method of claim 28, wherein the method elicits a T cell response to the chlamydia antigen.

31-32. (canceled)

33. The method of claim 28, wherein the method elicits an antibody response to the chlamydia antigen.

34-35. (canceled)

36. The method of claim 28, wherein the immunogenic composition is administered to the mammal at least two times.

37. The method of claim 28, wherein the mammal is at risk for infection with Chlamydia trachomatis.

38. The method of claim 28, wherein the mammal is infected with Chlamydia trachomatis.

39-54. (canceled)

55. The method of claim 28, wherein the composition comprises two or more chlamydia antigens.

56-61. (canceled)

62. An isolated nucleic acid comprising a nucleotide sequence encoding a chlamydia antigen selected from a CT209 polypeptide antigen, a CT253 polypeptide antigen, a CT425 polypeptide antigen, a CT497 polypeptide antigen, and a CT843 polypeptide antigen.

63. The nucleic acid of claim 62, wherein the nucleic acid further comprises a nucleotide sequence encoding a heterologous peptide fused to the chlamydia antigen.

64. The nucleic acid of claim 62, further comprising a pharmaceutically acceptable excipient.

65. (canceled)

66. A method for eliciting an immune response against chlamydia in a mammal, the method comprising administering to the mammal a composition comprising a nucleic acid, wherein the nucleic acid comprises a nucleotide sequence encoding a chlamydia antigen selected from a CT209 polypeptide antigen, a CT253 polypeptide antigen, a CT425 polypeptide antigen, a CT497 polypeptide antigen, and a CT843 polypeptide antigen.

67. A kit comprising an isolated chlamydia antigen selected from a CT209 polypeptide, a CT253 polypeptide antigen, a CT425 polypeptide antigen, a CT497 polypeptide antigen, and a CT843 polypeptide antigen.
